(19) United States
(12) Patent Application Publication
(10) Pub. No.: US 2025/0090507 A1
(43) Pub. Date: Mar. 20, 2025

(54) Methods of Treating Cancer With a B-RAF Inhibitor

(71) Applicant: MapKure, LLC, Wilmington, DE (US)

(72) Inventors: Lusong LUO, San Mateo, CA (US); Changyou ZHOU, San Mateo, CA (US); Brandon BEAGLE, San Mateo, CA (US); Nenad SARAPA, Stamford, CT (US)

(21) Appl. No.: 18/966,838

(22) Filed: Dec. 3, 2024

Related U.S. Application Data
(63) Continuation of application No. PCT/US2023/068119, filed on Jun. 8, 2023.
(60) Provisional application No. 63/350,332, filed on Jun. 8, 2022.

(51) Int. Cl. A61K 31/4375; A61P 35/00

(52) U.S. Cl. CPC A61K 31/4375; A61P 35/00

(57) ABSTRACT

Provided here was a method of treating a cancer in a subject in need thereof, comprising administering to said subject from about 5 mg to about 600 mg of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl)urea, or a pharmaceutically acceptable salt, tautomer, stereoisomer, enantiomer, isotopologue, solvate, or prodrug thereof.

### CROSS-REFERENCE TO RELATED APPLICATIONS

This application is a continuation of International Application No. PCT/US2023/068119 filed on Jun. 8, 2023, which claims priority to U.S. provisional application No. 63/350,332 filed on Jun. 8, 2022, the disclosures of each of which are hereby incorporated by reference in their entireties.

### FIELD

Provided herein are methods of treating cancer in a mammal. In particular, the method relates to the use of a B-Raf inhibitor, particularly 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea or a pharmaceutically acceptable salt, tautomer, stereoisomer, enantiomer, isotopologue, solvate, or prodrug thereof, for treating cancer.

### BACKGROUND

Effective treatment of hyperproliferative disorders including cancer is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes include abnormalities in signal transduction pathways and response to factors which differ from those found in normal cells.

Receptor tyrosine kinases (RTKs) catalyze phosphorylation of certain tyrosyl amino acid residues in various proteins, including themselves, which govern cell growth, proliferation, and differentiation.

Downstream of the several RTKs lie several signaling pathways, among them is the Ras-Raf-MEK-ERK kinase pathway. It is currently understood that activation of Ras GTPase proteins in response to growth factors, hormones, cytokines, etc. stimulates phosphorylation and activation of Raf kinases. These kinases then phosphorylate and activate the intracellular protein kinases MEK1 and MEK2, which in turn phosphorylate and activate other protein kinases, ERK1 and 2. This signaling pathway, also known as the mitogen-activated protein kinase (MAPK) pathway or cytoplasmic cascade, mediates cellular responses to growth signals. The ultimate function of this is to link receptor activity at the cell membrane with modification of cytoplasmic or nuclear targets that govern cell proliferation, differentiation, and survival.

The constitutive activation of this pathway is sufficient to induce cellular transformation. Dysregulated activation of the MAP kinase pathway due to aberrant receptor tyrosine kinase activation, Ras mutations or Raf mutations has frequently been found in human cancers, and represents a major factor determining abnormal growth control. In human malignances, Ras mutations are common, having been identified in about 30% of cancers. The Ras family of GTPase proteins (proteins which convert guanosine triphosphate to guanosine diphosphate) relay signals from activated growth factor receptors to downstream intracellular partners. Prominent among the targets recruited by active membrane-bound Ras are the Raf family of serine/threonine protein kinases. The Raf family is composed of three related kinases (A-, B- and C-Raf) that act as downstream effectors of Ras. Ras-medicated Raf activation in turn triggers activation of MEK1 and MEK2 (MAP/ERK kinases 1 and 2) which in turn phosphorylate ERK1 and ERK2 (extracellular signal-regulated kinases 1 and 2) on the tyrosine-185 and threonine-183. Activated ERK1 and ERK2 translocate and accumulate in the nucleus, where they can phosphorylate a variety of substrates, including transcription factors that control cellular growth and survival. Given the importance of the Ras/Raf/MEK/ERK pathway in the development of human cancers, the kinase components of the signaling cascade are merging as potentially important targets for the modulation of disease progression in cancer and other proliferative diseases.

Mutations in various Ras GTPases and the B-Raf kinase have been identified that can lead to sustained and constitutive activation of the MAPK pathway, ultimately resulting in increased cell division and survival. As a consequence of this, these mutations have been strongly linked with the establishment, development, and progression of a wide range of human cancers. The biological role of the Raf kinases, and specifically that of B-Raf, in signal transduction is described in Davies, H., et al., Nature (2002) 9:1-6; Garnett, M. J. & Marais, R., Cancer Cell (2004) 6:313-319; Zebisch, A. & Troppmair, J., Cell. Mol. Life Sci. (2006) 63:1314-1330; Midgley, R. S. & Kerr, D. J., Crit. Rev. Onc/Hematol. (2002) 44:109-120; Smith, R. A., et al., Curr. Top. Med. Chem. (2006) 6:1071-1089; and Downward, J., Nat. Rev. Cancer (2003) 3:1 1-22.

Naturally occurring mutations of the B-Raf kinase that activate MAPK pathway signaling have been found in a large percentage of human melanomas (Davies (2002) supra) and thyroid cancers (Cohen et al J. Nat. Cancer Inst. (2003) 95(8) 625-627 and Kimura et al Cancer Res. (2003) 63(7) 1454-1457), as well as at lower, but still significant, frequencies in the following: Barret's adenocarcinoma (Garnett et al., Cancer Cell (2004) 6 313-319 and Sommerer et al Oncogene (2004) 23(2) 554-558), billiary tract carcinomas (Zebisch et al., Cell. Mol. Life Sci. (2006) 63 1314-1330), breast cancer (Davies (2002) supra), cervical cancer (Moreno-Bueno et al Clin. Cancer Res. (2006) 12(12) 3865-3866), cholangiocarcinoma (Tannapfel et al Gut (2003) 52(5) 706-712), central nervous system tumors including primary CNS tumors such as glioblastomas, astrocytomas and ependymomas (Knobbe et al., Acta Neuropathol. (Berl.) (2004) 108(6) 467-470, Davies (2002) supra, and Garnett et al., Cancer Cell (2004) supra) and secondary CNS tumors (i.e., metastases to the central nervous system of tumors originating outside of the central nervous system), colorectal cancer, including large intestinal colon carcinoma (Yuen et al Cancer Res. (2002) 62(22) 6451-6455, Davies (2002) supra and Zebisch et al., Cell. Mol. Life Sci. (2006), gastric cancer (Lee et al Oncogene (2003) 22(44) 6942-6945), carcinoma of the head and neck including squamous cell carcinoma of the head and neck (Cohen et al J. Nat. Cancer Inst. (2003) 95(8) 625-627 and Weber et al Oncogene (2003) 22(30) 4757-4759), hematologic cancers including leukemias (Garnett et al., Cancer Cell (2004) supra, particularly acute lymphoblastic leukemia (Garnett et al., Cancer Cell (2004) supra and Gustafsson et al Leukemia (2005) 19(2) 310-312), acute myelogenous leukemia (AML) (Lee et al Leukemia (2004) 18(1) 170-172, and Christiansen et al Leukemia (2005) 19(12) 2232-2240), myelodysplasia syndromes (Christiansen et al Leukemia (2005) supra) and chronic myelogenous leukemia (Mizuchi et al Biochem. Biophys. Res. Commun. (2005) 326(3) 645-651); Hodgkin's lymphoma (Figl et al Arch. Dermatol. (2007) 143(4) 495-499), non-Hodgkin's lymphoma (Lee et al Br. J. Cancer (2003) 89(10) 1958-1960), megakaryoblastic leukemia (Eychene et al Oncogene (1995) 10(6) 1 159-1 165) and multiple myeloma (Ng et al Br. J. Haematol. (2003) 123(4) 637-645), hepatocellular carcinoma (Garnett et al., Cancer Cell (2004), lung cancer (Brose et al Cancer Res. (2002) 62(23) 6997-7000, Cohen et al J. Nat. Cancer Inst. (2003) supra and Davies (2002) supra), including small cell lung cancer (Pardo et al EMBO J. (2006) 25(13) 3078-3088) and non-small cell lung cancer (Davies (2002) supra), ovarian cancer (Russell & McCluggage J. Pathol. (2004) 203(2) 617-619 and Davies (2002) supr), endometrial cancer (Garnett et al., Cancer Cell (2004) supra, and Moreno-Bueno et al Clin. Cancer Res. (2006) supra), pancreatic cancer (Ishimura et al Cancer Lett. (2003) 199(2) 169-173), pituitary adenoma (De Martino et al J. Endocrinol. Invest. (2007) 30(1) RC1-3), prostate cancer (Cho et al Int. J. Cancer (2006) 1 19(8) 1858-1862), renal cancer (Nagy et al Int. J. Cancer (2003) 106(6) 980-981), sarcoma (Davies (2002) supra), and skin cancers (Rodriguez-Viciana et al., Science (2006) 31 1 (5765) 1287-1290 and Davies (2002) supra). Overexpression of c-Raf has been linked to AML (Zebisch et al., Cancer Res. (2006) 66(7) 3401-3408, and Zebisch (Cell. Mol. Life Sci. (2006)) and erythroleukemia (Zebisch et la., Cell. Mol. Life Sci. (2006).

By virtue of the role played by the Raf family kinases in these cancers and exploratory studies with a range of preclinical and therapeutic agents, including one selectively targeted to inhibition of B-Raf kinase activity (King A. J., et al., (2006) Cancer Res. 66:1 1 100-1 1 105), it is generally accepted that inhibitors of one or more Raf family kinases is useful for the treatment of such cancers or other condition associated with Raf kinase.

Mutation of B-Raf has also been implicated in other conditions, including cardio-facio cutaneous syndrome (Rodriguez-Viciana et al Science (2006) 31 1 (5765) 1287-1290) and polycystic kidney disease (Nagao et al Kidney Int. (2003) 63(2) 427-437).

Though there have been many recent advances in the treatment of cancer with compounds such as the B-Raf inhibitors, there remains a need for more effective and/or enhanced treatment of an individual suffering the effects of cancer, in particular, in cancers with MAPK pathway aberrations.

Citation or identification of any reference in this section is not to be construed as an admission that the reference is prior art to the present application.

### SUMMARY

Provided here is a method of treating a cancer in a subject in need thereof, comprising administering to said subject Compound A having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl)urea, or the structure of formula (I):

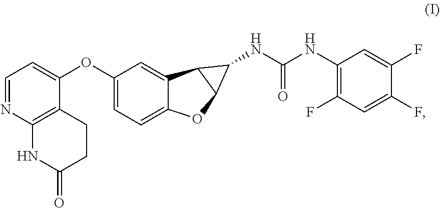 [SMILES: O=C1CCc2c(Oc3ccc4c(c3)[C@H]3[C@H](NC(=O)Nc5cc(F)c(F)cc5F)[C@H]3O4)ccnc2N1]

or a pharmaceutically acceptable salt, tautomer, stereoisomer, enantiomer, isotopologue, solvate, or prodrug thereof.

In one embodiment, the cancer is selected from the group consisting of colorectal cancer, pancreatic cancer, melanoma, non-small cell lung cancer, brain cancer, lung cancer, kidney cancer, bone cancer, liver cancer, bladder cancer, breast cancer, head and neck cancer, ovarian cancer, skin cancer, adrenal cancer, cervical cancer, lymphoma, thyroid tumor; preferably melanoma, ovarian cancer, and non-small cell lung cancer. In one embodiment, the cancer is melanoma. In one embodiment, the melanoma is cutaneous melanoma. In one embodiment, the melanoma is metastatic melanoma. In one embodiment, cancer is ovarian cancer. In one embodiment, the cancer is non-small cell lung cancer.

In one embodiment, the cancer is characterized by a mutation in a gene selected from the group consisting of RAS, NRAS, KRAS, RAF, BRAF, CRAF, ARAF, and their combination thereof; preferably RAS, NRAS, KRAS, RAF, BRAF, and their combination thereof; more preferably NRAS, KRAS, BRAF, and their combination thereof. In one embodiment, the cancer is characterized by a mutation selected from the group consisting of NRAS Q61R, NRAS Q61K, NRAS Q61L, NRAS G12S, NRAS G13R, KRAS G12A, KRAS G12C, KRAS G12D, KRAS G12V, BRAF V600E, BRAF fusion, and their combination thereof; preferably NRAS Q61R, NRAS Q61K, NRAS Q61L, KRAS G12D, KRAS G12V, BRAF V600E, BRAF fusion, and their combination thereof; more preferably NRAS Q61R, NRAS Q61K, NRAS Q61L, KRAS G12D, KRAS G12V, and their combination thereof. In one embodiment, the cancer is characterized by other MAPK pathway genomic aberration. In one embodiment, the other MAPK pathway genomic aberration is RASAl splice isoform.

In one embodiment, Compound A is administered one to three times a day. In one embodiment, Compound A is administered once a day. In one embodiment, Compound A is administered at about 40 mg, about 50 mg, about 60 mg, about 70 mg, about 80 mg, about 90 mg, about 100 mg, about 110 mg, about 120 mg, about 130 mg, about 140 mg, about 150 mg, or about 160 mg per day. In one embodiment, Compound A is administered at about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg per day. In one embodiment, Compound A is administered at about 5 mg, about 10 mg, about 15 mg, about 20 mg, about 25 mg, about 30 mg, about 35 mg, about 40 mg, about 45 mg, about 50 mg, about 55 mg, or about 60 mg per day. In one embodiment, Compound A is administered at about 5 mg, about 10 mg, about 15 mg, about 25 mg, about 40 mg, or about 60 mg per day. In one embodiment, Compound A is administered at about 40 mg, or about 60 mg per day. In one embodiment, Compound A is administered at about 40 mg per day. In one embodiment, Compound A is administered at about 60 mg per day.

In one embodiment, the method as described herein provides a plasma Compound A AUC₈ₕ between about 2,128 ng*h/ml and about 3,192 ng*h/ml in the subject. In one embodiment, the method as described herein provides a plasma Compound A AUC₈ₕ between about 4,576 ng*h/ml and about 6,864 ng*h/ml in the subject. In one embodiment, the method as described herein provides a plasma Compound A AUC₈ₕ between about 7,944 ng*h/ml and about 11,916 ng*h/ml in the subject. In one embodiment, the method as described herein provides a plasma Compound A AUC₈ₕ between about 9,840 ng*h/ml and about 14,760 ng*h/ml in the subject. In one embodiment, the method as described herein provides a plasma Compound A AUC₈ₕ between about 12,640 ng*h/ml and about 18,960 ng*h/ml in the subject. In one embodiment, the method as described herein provides a plasma Compound A AUC₈ₕ between about 30,000 ng*h/ml and about 45,000 ng*h/ml in the subject.

In one embodiment, the subject achieves a stable disease, a partial response, or a complete response. In one embodiment, the subject does not experience a progressive disease.

### BRIEF DESCRIPTION OF DRAWINGS

FIG. 1 describes the best change (%) from baseline in sum of diameters per RECIST 1.1 (efficacy in total 37 evaluable patients).

FIG. 2 describes the dose escalation.

FIG. 3 provides the preliminary mean PK profiles for all cohorts for C1D1.

FIG. 4 provides the preliminary mean PK profiles for all cohorts for C2D1.

### DETAILED DESCRIPTION

### Definitions

As used herein, “Compound A” refers to the compound having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl)urea, or the structure of formula (I):

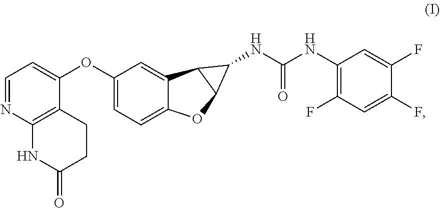 [SMILES: O=C1CCc2c(Oc3ccc4c(c3)[C@H]3[C@H](NC(=O)Nc5cc(F)c(F)cc5F)[C@H]3O4)ccnc2N1]

or a pharmaceutically acceptable salt, tautomer, stereoisomer, enantiomer, isotopologue, solvate, or prodrug thereof. Compound A is disclosed and claimed, along with pharmaceutically acceptable salts thereof, and also as solvates thereof, as being useful as an inhibitor of BRAF activity, particularly in treatment of cancer, in WO2014206343 and WO2020151756, the entire disclosure of which are incorporated herein by reference. Compound A is compound 1.49 in WO2014206343 and Compound 1 in WO2020151756. Compound A can be prepared as described in WO2014206343and WO2020151756. In one embodiment, Compound A is a hydrate. Unless specifically stated, “Compound A” as used herein refers to Compound A or a pharmaceutically acceptable salt, tautomer, stereoisomer, enantiomer, isotopologue, solvate, or prodrug thereof.

In one embodiment, the solid form of Compound A is used for the treatment provided herein. In one embodiment, a crystal form of Compound A is used for the treatment provided herein. In one embodiment, the amorphous form of Compound A is used for the treatment provided herein. In one embodiment, the freebase of Compound A is used for the treatment provided herein. In one embodiment, the HCl salt of Compound A is used for the treatment provided herein. In one embodiment, Form I of Compound A described in Example 10 of WO2020151756 is used for the treatment provided herein.

A BRAF inhibitor is a chemical or drug that inhibits the mitogen-activated protein kinase enzymes BRAF. They can be used to affect the MAPK/ERK pathway. For example, BRAF inhibitors include, but are not limited to, Compound A, lifirafenib, dabrafenib, vemurafenib, encorafenib, LY3009120, PLX8394, LXH254, MLN2480, Raf709, TAK632, and PLX7904.

As used herein the term “neoplasm” refers to an abnormal growth of cells or tissue and is understood to include benign, i.e., non-cancerous growths, and malignant, i.e., cancerous growths. The term “neoplastic” means of or related to a neoplasm.

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

By the term “treating” and derivatives thereof as used herein, is meant therapeutic therapy. In reference to a particular condition, treating means: (1) to ameliorate the condition or one or more of the biological manifestations of the condition, (2) to interfere with (a) one or more points in the biological cascade that leads to or is responsible for the condition or (b) one or more of the biological manifestations of the condition (3) to alleviate one or more of the symptoms, effects or side effects associated with the condition or one or more of the symptoms, effects or side effects associated with the condition or treatment thereof, or (4) to slow the progression of the condition or one or more of the biological manifestations of the condition.

As used herein, “prevention” is understood to refer to the prophylactic administration of a drug to substantially diminish the likelihood or severity of a condition or biological manifestation thereof, or to delay the onset of such condition or biological manifestation thereof. The skilled artisan would appreciate that “prevention” is not an absolute term. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that elicit the biological or medical response of a tissue, system, animal, or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compound A disclosed herein may contain one or more chiral atoms, or may otherwise be capable of existing as enantiomers. Accordingly, the compounds of this invention include mixtures of enantiomers as well as purified enantiomers or enantiomerically enriched mixtures. Also, it is understood that all tautomers and mixtures of tautomers are included within the scope of Compound A.

As used herein, the term “solvate” refers to a complex of variable stoichiometry formed by a solute (in this invention, compounds of formula (I) or a salt thereof and a solvent. Also, it is understood that Compound A may be presented, separately or both, as solvates. Such solvents for the purpose of the invention may not interfere with the biological activity of the solute. Examples of suitable solvents include, but are not limited to, water, methanol, dimethylsulforide. ethanol and acetic acid. In one embodiment, the solvent used is a pharmaceutically acceptable solvent. Examples of suitable pharmaceutically acceptable solvents include, without limitation, water, ethanol and acetic acid. In another embodiment, the solvent used is water (i.e., a hydrate).

Compound A may have the ability to crystallize in more than one form, a characteristic, which is known polymorphism, and it is understood that such polymorphic forms (“polymorphs”) are within the scope of Compound A. Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percentages of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 2θ (or ±0.2 degrees 2θ) while still describing the particular XRPD peak.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds provided herein include, but are not limited to those well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center is substantially free of the opposite enantiomer of the compound. A stereomerically pure compound having two chiral centers is substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

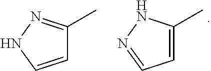

As readily understood by one skilled in the art, a wide variety of functional groups and other structures may exhibit tautomerism and all tautomers of the compounds provided herein are within the scope of the present invention.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (14° C.), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

The term “subject” includes an animal, including, but not limited to, an animal such a cow, monkey, horse, sheep, pig, chicken, turkey, quail, cat, dog, mouse, rat, rabbit, or guinea pig, in one embodiment a mammal, in another embodiment a human.

While it is possible that, for use in therapy, Compound A, may be administered as the raw chemical, it is possible to present the active ingredient as a pharmaceutical composition. Accordingly, the invention further provides pharmaceutical compositions, which include a Compound A, and one or more pharmaceutically acceptable carriers, diluents, or excipients. Compound A is as described above. The carrier(s), diluent(s) or excipient(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical composition including admixing a Compound A with one or more pharmaceutically acceptable carriers, diluents, or excipients. Such elements of the pharmaceutical compositions utilized may be presented in separate pharmaceutical combinations or formulated together in one pharmaceutical composition. Accordingly, the invention further provides a pharmaceutical composition containing Compound A and one or more pharmaceutically acceptable carriers, diluents, or excipients. Compound A described above may be utilized in any of the compositions described above.

Pharmaceutical compositions may be presented in unit dose forms containing a predetermined amount of active ingredient per unit dose. As is known to those skilled in the art, the amount of active ingredient per dose depends on the condition being treated, the route of administration and the age, weight and condition of the patient. Preferred unit dosage compositions are those containing a daily dose or sub-dose, or an appropriate fraction thereof, of an active ingredient. Furthermore, such pharmaceutical compositions may be prepared by any of the methods well known in the pharmacy art.

Compound A may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It is appreciated that the preferred route may vary with, for example, the condition of the recipient of the combination and the cancer to be treated. It will also be appreciated that each of the agents administered may be administered by the same or different routes and that the Compound A may be compounded together in a pharmaceutical composition.

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Unless otherwise defined, in all dosing protocols described herein, the regimen of compounds administered does not have to commence with the start of treatment and terminate with the end of treatment, it is only required that the number of consecutive days in which both compounds are administered and the optional number of consecutive days in which only one of the component compounds is administered, or the indicated dosing protocol—including the amount of compound administered, occur at some point during the course of treatment.

Compound A may be employed in combination in accordance with the disclosure by administration simultaneously in a unitary pharmaceutical composition including both compounds.

Furthermore, it does not matter if the compounds are administered in the same dosage form, e.g., one compound may be administered topically and the other compound may be administered orally. Suitably, both compounds are administered orally.

Unless otherwise defined, in all dosing protocols described herein, the regimen of compounds administered does not have to commence with the start of treatment and terminate with the end of treatment, it is only required that the number of consecutive days in which both compounds are administered and the optional number of consecutive days in which only one of the component compounds is administered, or the indicated dosing protocol—including the amount of compound administered, occur at some point during the course of treatment.

By the term “kit” “or kit of parts” as used herein is meant the pharmaceutical composition or compositions that are used to administer Compound A according to the disclosure. In one embodiment, the kit can contain Compound A in a single pharmaceutical composition, such as a tablet, or in separate pharmaceutical compositions. In one aspect, there is provided a kit of parts comprising components: Compound A in association with a pharmaceutically acceptable excipients, diluents, or carrier. The kit can also be provided with instruction, such as dosage and administration instructions. Such dosage and administration instructions can be of the kind that are provided to a doctor, for example by a drug product label, or they can be of the kind that are provided by a doctor, such as instructions to a patient.

The term “dose” as used herein is understood to mean a dose that is intended to either slowly raise plasma or blood concentration levels of the compound to a therapeutically effective level, or to maintain such a therapeutically effective level.

In certain embodiments, the treatment of a cancer may be assessed by Response Evaluation Criteria in Solid Tumors (RECIST 1.1) (see Thereasse P., et al. New Guidelines to Evaluate the Response to Treatment in Solid Tumors. J. of the National Cancer Institute; 2000; (92) 205-216 and Eisenhauer E.A., Therasse P., Bogaerts J., et al. New response evaluation criteria in solid tumors: Revised RECIST guideline (version 1.1). European J. Cancer; 2009; (45) 228-247). Overall responses for all possible combinations of tumor responses in target and non-target lesions with or without the appearance of new lesions are as follows:

| Target lesions | Non-target lesions | New lesions | Overall response |
|---|---|---|---|
| CR | CR | No | CR |
| CR | Incomplete response/SD | No | PR |
| PR | Non-PD | No | PR |
| SD | Non-PD | No | SD |
| PD | Any | Yes or no | PD |
| Any | PD | Yes or no | PD |
| Any | Any | Yes | PD |

CR = complete response; PR = partial response; SD = stable disease; and PD = progressive disease.

With respect to the evaluation of target lesions, complete response (CR) is the disappearance of all target lesions, partial response (PR) is at least a 30% decrease in the sum of the longest diameter of target lesions, taking as reference the baseline sum longest diameter, progressive disease (PD) is at least a 20% increase in the sum of the longest diameter of target lesions, taking as reference the smallest sum longest diameter recorded since the treatment started or the appearance of one or more new lesions and stable disease (SD) is neither sufficient shrinkage to qualify for partial response nor sufficient increase to qualify for progressive disease, taking as reference the smallest sum longest diameter since the treatment started.

With respect to the evaluation of non-target lesions, complete response (CR) is the disappearance of all non-target lesions and normalization of tumor marker level; incomplete response/stable disease (SD) is the persistence of one or more non-target lesion(s) and/or the maintenance of tumor marker level above the normal limits, and progressive disease (PD) is the appearance of one or more new lesions and/or unequivocal progression of existing non-target lesions.

The procedures, conventions, and definitions described below provide guidance for implementing the recommendations from the Response Assessment for Neuro-Oncology (RANO) Working Group regarding response criteria for high-grade gliomas (Wen P., Macdonald, D R., Reardon, D A., et al. Updated response assessment criteria for high-grade gliomas: Response assessment in neuro-oncology working group. J Clin Oncol 2010; 28:1963-1972). Primary modifications to the RANO criteria for Criteria for Time Point Responses (TPR) can include the addition of operational conventions for defining changes in glucocorticoid dose, and the removal of subjects' clinical deterioration component to focus on objective radiologic assessments. The baseline MRI scan is defined as the assessment performed at the end of the post-surgery rest period, prior to initiating or re-initiating compound treatment. The baseline MRI is used as the reference for assessing complete response (CR) and partial response (PR). Whereas, the smallest SPD (sum of the products of perpendicular diameters) obtained either at baseline or at subsequent assessments are designated the nadir assessment and utilized as the reference for determining progression. For the 5 days preceding any protocol-defined MRI scan, subjects receive either no glucocorticoids or are on a stable dose of glucocorticoids. A stable dose is defined as the same daily dose for the 5 consecutive days preceding the MRI scan. If the prescribed glucocorticoid dose is changed in the 5 days before the baseline scan, a new baseline scan is required with glucocorticoid use meeting the criteria described above. The following definitions are used.

Measurable Lesions: Measurable lesions are contrast-enhancing lesions that can be measured bi-dimensionally. A measurement is made of the maximal enhancing tumor diameter (also known as the longest diameter, LD). The greatest perpendicular diameter is measured on the same image. The cross hairs of bi-dimensional measurements should cross and the product of these diameters are calculated.

Minimal Diameter: T1-weighted image in which the sections are 5 mm with 1 mm skip. The minimal LD of a measurable lesion is set as 5 mm by 5 mm. Larger diameters may be required for inclusion and/or designation as target lesions. After baseline, target lesions that become smaller than the minimum requirement for measurement or become no longer amenable to bi-dimensional measurement are recorded at the default value of 5 mm for each diameter below 5 mm. Lesions that disappear are recorded as 0 mm by 0 mm.

Multicentric Lesions: Lesions that are considered multicentric (as opposed to continuous) are lesions where there is normal intervening brain tissue between the two (or more) lesions. For multicentric lesions that are discrete foci of enhancement, the approach is to separately measure each enhancing lesion that meets the inclusion criteria. If there is no normal brain tissue between two (or more) lesions, they are considered the same lesion.

Nonmeasurable Lesions: All lesions that do not meet the criteria for measurable disease as defined above are considered non-measurable lesions, as well as all non-enhancing and other truly nonmeasurable lesions. Nonmeasurable lesions include foci of enhancement that are less than the specified smallest diameter (i.e., less than 5 mm by 5 mm), non-enhancing lesions (e.g., as seen on T1-weighted post-contrast, T2-weighted, or fluid-attenuated inversion recovery (FLAIR) images), hemorrhagic or predominantly cystic or necrotic lesions, and leptomeningeal tumor. Hemorrhagic lesions often have intrinsic T1-weighted hyperintensity that could be misinterpreted as enhancing tumor, and for this reason, the pre-contrast T1-weighted image may be examined to exclude baseline or interval sub-acute hemorrhage.

At baseline, lesions are classified as follows: Target lesions: Up to 5 measurable lesions can be selected as target lesions with each measuring at least 10 mm by 5 mm, representative of the subject's disease; Non-target lesions: All other lesions, including all nonmeasurable lesions (including mass effects and T2/FLAIR findings) and any measurable lesion not selected as a target lesion. At baseline, target lesions are to be measured as described in the definition for measurable lesions and the SPD of all target lesions is to be determined. The presence of all other lesions is to be documented. At all post-treatment evaluations, the baseline classification of lesions as target and non-target lesions are maintained and lesions are documented and described in a consistent fashion over time (e.g., recorded in the same order on source documents and eCRFs). All measurable and nonmeasurable lesions must be assessed using the same technique as at baseline (e.g., subjects should be imaged on the same MRI scanner or at least with the same magnet strength) for the duration of the study to reduce difficulties in interpreting changes. At each evaluation, target lesions are measured and the SPD calculated. Non-target lesions are assessed qualitatively and new lesions, if any, are documented separately. At each evaluation, a time point response is determined for target lesions, non-target lesions, and new lesion. Tumor progression can be established even if only a subset of lesions is assessed. However, unless progression is observed, objective status (stable disease, PR or CR) can only be determined when all lesions are assessed.

Confirmation assessments for overall time point responses of CR and PR are performed at the next scheduled assessment, but confirmation may not occur if scans have an interval of <28 days. Best response, incorporating confirmation requirements, are derived from the series of time points.

As used herein, all amounts specified for Compound A are indicated as the amount of free or unsalted compound.

### Kits

Provided herein is a kit comprising a compound provided herein and means for monitoring patient response to administration of said compound provided herein. In certain embodiments, the patient has colorectal cancer, pancreatic cancer, melanoma, non-small cell lung cancer, brain cancer, lung cancer, kidney cancer, bone cancer, liver cancer, bladder cancer, breast, head and neck cancer, ovarian cancer, skin cancer, adrenal cancer, cervical cancer, lymphoma, thyroid tumor, and their complications; preferably melanoma, ovarian cancer, and non-small cell lung cancer. In particular embodiments, the patient response measured is inhibition of disease progression, inhibition of tumor growth, reduction of primary and/or secondary tumor(s), relief of tumor-related symptoms, improvement in quality of life, delayed appearance of primary and/or secondary tumors, slowed development of primary and/or secondary tumors, decreased occurrence of primary and/or secondary tumors, slowed or decreased severity of secondary effects of disease, arrested tumor growth or regression of tumor.

In other embodiments, provided herein are kits comprising a compound provided herein and means for measuring the amount of inhibition of B-RAF, KRAS, or MEK in a patient. In certain embodiments, the kits comprise means for measuring inhibition of B-RAF or MEK in circulating plasma, blood, or tumor cells and/or skin biopsies or tumor biopsies/aspirates of a patient. In certain embodiments, provided herein are kits comprising a compound provided herein and means for measuring the amount of inhibition of B-RAF, KRAS or MEK before, during and/or after administration of a compound provided herein. In certain embodiments, the patient has colorectal cancer, pancreatic cancer, melanoma, ovarian cancer, or non-small cell lung cancer.

In certain embodiments, the kits provided herein comprise an amount of a compound provided herein effective for treating or preventing colorectal cancer, pancreatic cancer, melanoma, non-small cell lung cancer, brain cancer, lung cancer, kidney cancer, bone cancer, liver cancer, bladder cancer, breast, head and neck cancer, ovarian cancer, skin cancer, adrenal cancer, cervical cancer, lymphoma, thyroid tumor, and their complications; preferably melanoma, ovarian cancer, and non-small cell lung cancer. In certain embodiments, the kits provided herein comprise an amount of a compound provided herein effective for treating or preventing colorectal cancer, pancreatic cancer, melanoma, ovarian cancer, or non-small cell lung cancer.

In certain embodiments, the kits provided herein further comprise instructions for use, such as for administering a compound provided herein and/or monitoring patient response to administration of the compound.

### Method of Treatment

Provided herein is a method of treating a cancer in a subject in need thereof, comprising administering to said subject Compound A.

In one embodiment, the cancer is selected from the group consisting of colorectal cancer, pancreatic cancer, melanoma, non-small cell lung cancer, brain cancer, lung cancer, kidney cancer, bone cancer, liver cancer, bladder cancer, breast, head and neck cancer, ovarian cancer, skin cancer, adrenal cancer, cervical cancer, lymphoma, thyroid tumor, and their complications; preferably melanoma, ovarian cancer, and non-small cell lung cancer.

In one embodiment, the cancer is characterized by a mutation in a gene selected from the group consisting of RAS, NRAS, KRAS, RAF, BRAF, CRAF, ARAF, and their combination thereof; preferably RAS, NRAS, KRAS, RAF, BRAF, and their combination thereof, more preferably NRAS, KRAS, BRAF, and their combination thereof.

In one embodiment, the cancer is characterized by a mutation selected from the group consisting of NRAS Q61R, NRAS Q61K, NRAS Q61L, NRAS G12S, NRAS G13R, KRAS G12A, KRAS G12C, KRAS G12D, KRAS G12V, BRAF V600E, BRAF fusion, and their combination thereof; preferably NRAS Q61R, NRAS Q61K, NRAS Q61L, KRAS G12D, KRAS G12V, BRAF V600E, BRAF fusion, and their combination thereof; more preferably NRAS Q61R, NRAS Q61K, NRAS Q61L, KRAS G12D, KRAS G12V, and their combination thereof.

In one embodiment, the cancer is characterized by other MAPK pathway genomic aberration. In one embodiment, the other MAPK pathway genomic aberration is RAS Al splice isoform.

In one embodiment, the cancer is characterized by a mutation in a gene selected from the group consisting of ARAF, BRAF, RAF1, KRAS, HRAS, NF1, MAP2K1, MAP2K2, MAPK1, and their combination thereof.

In one embodiment, the cancer is characterized by a mutation selected from the group consisting of BRAF N20T, BRAF A33T, BRAF S36A, BRAF V47_G393del, BRAF V47_G327del, BRAF V47_D380del, BRAF V47_M438del, BRAF N49I, BRAF M53I, BRAF L64I, BRAF G69S, BRAF A81_D380del, BRAF A81_M438del, BRAF G104E, BRAF T119S, BRAF P141L, BRAF S151A, BRAF P162S, BRAF V169_G327del, BRAF V169_D380del, BRAF R188T, BRAF Q201H, BRAF G203_G393del, BRAF K205Q, BRAF V226L, BRAF E228V, BRAF R239Q, BRAF T241P, BRAF T241M, BRAF L245F, BRAF A246P, BRAF F247L, BRAF Q257R, BRAF Q257H, BRAF G258V, BRAF F259L, BRAF Q262R, BRAF H269Y, BRAF R271H, BRAF E275K, BRAF D287H, BRAF F294L, BRAF T310I, BRAF A320T, BRAF 1326V, BRAF P341S, BRAF R347*, BRAF P348T, BRAF S363F, BRAF S364L, BRAF P367S, BRAF P367R, BRAF P367L, BRAF D380H, BRAF R389C, BRAF T401I, BRAF A404Cfs*9, BRAF P407L, BRAF S419Y, BRAF G421V, BRAF R444W, BRAF D448Y, BRAF D449Y, BRAF W450*, BRAF W450L, BRAF E451K, BRAF E451Q, BRAF P453T, BRAF V459L, BRAF R462E, BRAF R462K, BRAF R462I, BRAF 1463T, BRAF 1463S, BRAF G464I, BRAF G464R, BRAF G464E, BRAF G464A, BRAF G464V, BRAF S465D, BRAF S465E, BRAF S465A, BRAF G466R, BRAF G466E, BRAF G466A, BRAF G466V, BRAF S467A, BRAF S467L, BRAF F468C, BRAF G469L, BRAF G469del, BRAF G469S, BRAF G469R, BRAF G469E, BRAF G469A, BRAF G469V, BRAF T470K, BRAF V471I, BRAF V471F, BRAF Y472dup, BRAF Y472S, BRAF Y472C, BRAF G478C, BRAF K483E, BRAF K483M, BRAF L485_P490del, BRAF L485Y, BRAF L485_P490delins Y, BRAF L485S, BRAF L485W, BRAF L485F, BRAF L485_P490delinsF, BRAF N486_Q494del, BRAF N486del, BRAF N486_T488del, BRAF N486_T491del, BRAF N486_L495del, BRAF N486D, BRAF N486_V487del, BRAF N486_P490del, BRAF N486_A489delinsK, BRAF N486_T491delinsK, BRAF V487_P490del, BRAF V487_P492delinsA, BRAF T488_P492del, BRAF T488_Q493delinsK, BRAF A489_P490del, BRAF P490del, BRAF P490_Q494del, BRAF K499E, BRAF K499N, BRAF E501K, BRAF E501G, BRAF V504_R506dup, BRAF V504I, BRAF L505F, BRAF L505H, BRAF R509G, BRAF R509H, BRAF L514V, BRAF M517I, BRAF Q524L, BRAF L525R, BRAF T529M, BRAF T529N, BRAF T529I, BRAF W531C, BRAF G534D, BRAF Y538H, BRAF R558Q, BRAF G563D, BRAF H568D, BRAF H574N, BRAF H574Y, BRAF H574Q, BRAF N581D, BRAF N581Y, BRAF N581T, BRAF N581S, BRAF N581I, BRAF N581K, BRAF 1582M, BRAF F583C, BRAF L584F, BRAF H585Y, BRAF E586K, BRAF D587A, BRAF D587G, BRAF D587E, BRAF V590I, BRAF V590G, BRAF I592V, BRAF 1592M, BRAF G593D, BRAF D594N, BRAF D594H, BRAF D594Y, BRAF D594_T599dup, BRAF D594A, BRAF D594G, BRAF D594V, BRAF D594E, BRAF F595L, BRAF F595S, BRAF G596S, BRAF G596R, BRAF G596C, BRAF G596D, BRAF G596V, BRAF L597S, BRAF L597V, BRAF L597Q, BRAF L597P, BRAF L597R, BRAF A598T, BRAF A598S, BRAF A598V, BRAF A598_T599insARC, BRAF A598_T599insV, BRAF T599dup, BRAF T599A, BRAF T599K, BRAF T599R, BRAF T599I, BRAF T599_V600insTT, BRAF T599_V600insS, BRAF T599_V600insETT, BRAF T599_V600insEAT, BRAF V600_K601delinsEN, BRAF V600_S605delinsEISRWR, BRAF V600K, BRAF V600R, BRAF V600Q, BRAF V600dup, BRAF V600delinsYM, BRAF V600M, BRAF V600L, BRAF V600D, BRAF V600_K601delinsE, BRAF V600E, BRAF V600A, BRAF V600G, BRAF K601del, BRAF K601Q, BRAF K601E, BRAF K601_W604del, BRAF K601T, BRAF K601I, BRAF K601_S602delinsNT, BRAF K601N, BRAF S602T, BRAF S602Y, BRAF S602F, BRAF R603* BRAF W604del, BRAF W604R, BRAF W604G, BRAF S605A, BRAF S605F, BRAF S605E, BRAF S605G, BRAF S605N, BRAF S605I, BRAF G606W, BRAF G606E, BRAF G606A, BRAF G606V, BRAF S607P, BRAF S607F, BRAF H608R, BRAF Q609E, BRAF Q609L, BRAF Q609H, BRAF E611D, BRAF L613F, BRAF G615R, BRAF L618F, BRAF W619R, BRAF S637*, BRAF V639I, BRAF E648Q, BRAF Y656D, BRAF R671Q, BRAF P676S, BRAF L678I, BRAF V681I, BRAF E695K, BRAF K698R, BRAF L711F, BRAF A712T, BRAF R719S, BRAF H725Y, BRAF A728V, BRAF P731T, BRAF P731S, BRAF P731L, BRAF A762E, BRAF A762V, and their combination thereof.

In one embodiment, the cancer is characterized by a mutation selected from the group consisting of KIAA1549-BRAF fusion, BCAS1-BRAF fusion, CCDC6-BRAF fusion, CDC42BPB-BRAF fusion, FAM131B-BRAF fusion, FXR1-BRAF fusion, GIT2-BRAF fusion, KLHL7-BRAF fusion, RNF130-BRAF fusion, TMEM106B-BRAF fusion, MKRN1-BRAF fusion, AGAP3-BRAF fusion, AGK-BRAF fusion, AKAP9-BRAF fusion, ARMC10-BRAF fusion, CUL1-BRAF fusion, GTF2I-BRAF fusion, PAPSS1-BRAF fusion, PCBP2-BRAF fusion, PPFIBP2-BRAF fusion, SND1-BRAF fusion, TRIM24-BRAF fusion, ZKSCAN1-BRAF fusion, SEPT3-BRAF fusion, and their combination thereof.

In one embodiment, the cancer is characterized by a mutation selected from the group consisting of NRAS G12A, NRAS G12C, NRAS G12D, NRAS G12N, NRAS G12P, NRAS G12R, NRAS G12S, NRAS G12V, NRAS G12Y, NRAS G13A, NRAS G13C, NRAS G13D, NRAS G13E, NRAS G13N, NRAS G13R, NRAS G13S, NRAS G13V, NRAS A18T, NRAS 124N, NRAS P34L, NRAS Y40*, NRAS Q43*, NRAS T50I, NRAS T58I, NRAS A59G, NRAS A59D, NRAS A59T, NRAS G60E, NRAS G60R, NRAS Q61E, NRAS Q61H, NRAS Q61H, NRAS Q61K, NRAS Q61L, NRAS Q61L, NRAS Q61P, NRAS Q61R, NRAS Q61R, NRAS Q61R, NRAS Q61*, NRAS E63K, NRAS Y64D, NRAS S65C, NRAS R68S, NRAS S89A, NRAS G115Efs*46, NRAS E132K, NRAS K135N, NRAS A146P, NRAS A146T, NRAS A146V, NRAS E162*, and their combination thereof.

In some embodiments, the cancer harbors the mutations as described herein. In some embodiments, the subject with the cancer harbors the mutations as described herein.

In one embodiment, the cancer is melanoma. In one embodiment, the melanoma is cutaneous melanoma. In one embodiment, the melanoma is metastatic melanoma. In one embodiment, the cancer is non-small cell lung cancer. In one embodiment, the cancer is colorectal cancer. In one embodiment, the cancer is ovarian cancer.

In one embodiment, the amount of Compound A (based on weight of unsalted/unsolvated amount) administered per day is an amount selected from about 5 mg to about 600 mg. In one embodiment, the amount is selected from about 10 mg to about 500 mg. In one embodiment, the amount is selected from about 20 mg to about 400 mg. In one embodiment, the amount is selected from about 30 mg to about 200 mg. In one embodiment, the amount is selected from about 40 mg to about 100 mg. For example, Compound A is administered per day at about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg per day. In one embodiment, Compound A is administered per day at about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg per day. In one embodiment, Compound A is administered per day at about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg per day. In one embodiment, Compound A is administered per day at about 40 mg, about 45 mg, about 50 mg, about 55 mg, about 60 mg, about 65 mg, about 70 mg, about 75 mg, or about 80 mg per day. In one embodiment, Compound A is administered per day at about 5 mg, about 10 mg, about 15 mg, about 25 mg, about 40 mg, or about 60 mg per day. In one embodiment, Compound A is administered at about 40 mg, or about 60 mg per day. In one embodiment, Compound A is administered at about 40 mg per day. In one embodiment, Compound A is administered per day at about 60 mg per day.

In one embodiment, Compound A is administered one to three times a day. In one embodiment, Compound A is administered three times a day. In one embodiment, Compound A is administered twice a day. In one embodiment, Compound A is administered once a day.

In one embodiment, the method as described herein provides a plasma Compound A AUC₈ₕ between about 2,000 ng*h/ml and about 3,200 ng*h/ml in the subject. In one embodiment, the method as described herein provides a plasma Compound A AUC₈ₕ between about 2,128 ng*h/ml and about 3,192 ng*h/ml in the subject. In one embodiment, the method as described herein provides a plasma Compound A AUC₈ₕ between about 2,400 ng*h/ml and about 2,900 ng*h/ml in the subject.

In one embodiment, the method as described herein provides a plasma Compound A AUC₈ₕ between about 4,600 ng*h/ml and about 6,900 ng*h/ml in the subject. In one embodiment, the method as described herein provides a plasma Compound A AUC₈ₕ between about 4,576 ng*h/ml and about 6,864 ng*h/ml in the subject. In one embodiment, the method as described herein provides a plasma Compound A AUC₈ₕ between about 5,100 ng*h/ml and about 6,300 ng*h/ml in the subject.

In one embodiment, the method as described herein provides a plasma Compound A AUC₈ₕ between about 8,000 ng*h/ml and about 12,000 ng*h/ml in the subject. In one embodiment, the method as described herein provides a plasma Compound A AUC₈ₕ between about 7,944 ng*h/ml and about 11,916 ng*h/ml in the subject. In one embodiment, the method as described herein provides a plasma Compound A AUC₈ₕ between about 8,900 ng*h/ml and about 10,900 ng*h/ml in the subject.

In one embodiment, the method as described herein provides a plasma Compound A AUC₈ₕ between about 10,000 ng*h/ml and about 14,800 ng*h/ml in the subject. In one embodiment, the method as described herein provides a plasma Compound A AUC₈ₕ between about 9,840 ng*h/ml and about 14,760 ng*h/ml in the subject. In one embodiment, the method as described herein provides a plasma Compound A AUC₈ₕ between about 11,100 ng*h/ml and about 13,500 ng*h/ml in the subject.

In one embodiment, the method as described herein provides a plasma Compound A AUC₈ₕ between about 12,700 ng*h/ml and about 19,000 ng*h/ml in the subject. In one embodiment, the method as described herein provides a plasma Compound A AUC₈ₕ between about 12,640 ng*h/ml and about 18,960 ng*h/ml in the subject. In one embodiment, the method as described herein provides a plasma Compound A AUC₈ₕ between about 14,200 ng*h/ml and about 17,400 ng*h/ml in the subject.

In one embodiment, the method as described herein provides a plasma Compound A AUC₈ₕ between about 30,000 ng*h/ml and about 45,000 ng*h/ml in the subject. In one embodiment, the method as described herein provides a plasma Compound A AUC₈ₕ between about 33,800 ng*h/ml and about 41,300 ng*h/ml in the subject.

In one embodiment, the method as described herein provides a plasma Compound A AUC₈ₕ between about 2,000 ng*h/ml and about 3,200 ng*h/ml in the subject receiving Compound A treatment at about 5 mg/day. In one embodiment, the method as described herein provides a plasma Compound A AUC₈ₕ between about 2,128 ng*h/ml and about 3,192 ng*h/ml in the subject receiving Compound A treatment at about 5 mg/day. In one embodiment, the method as described herein provides a plasma Compound A AUC₈ₕ between about 2,400 ng*h/ml and about 2,900 ng*h/ml in the subject receiving Compound A treatment at about 5 mg/day.

In one embodiment, the method as described herein provides a plasma Compound A AUC₈ₕ between about 4,600 ng*h/ml and about 6,900 ng*h/ml in the subject receiving Compound A treatment at about 10 mg/day. In one embodiment, the method as described herein provides a plasma Compound A AUC₈ₕ between about 4,576 ng*h/ml and about 6,864 ng*h/ml in the subject receiving Compound A treatment at about 10 mg/day. In one embodiment, the method as described herein provides a plasma Compound A AUC₈ₕ between about 5,100 ng*h/ml and about 6,300 ng*h/ml in the subject receiving Compound A treatment at about 10 mg/day.

In one embodiment, the method as described herein provides a plasma Compound A AUC₈ₕ between about 8,000 ng*h/ml and about 12,000 ng*h/ml in the subject receiving Compound A treatment at about 15 mg/day. In one embodiment, the method as described herein provides a plasma Compound A AUC₈ₕ between about 7,944 ng*h/ml and about 11,916 ng*h/ml in the subject receiving Compound A treatment at about 15 mg/day. In one embodiment, the method as described herein provides a plasma Compound A AUC₈ₕ between about 8,900 ng*h/ml and about 10,900 ng*h/ml in the subject receiving Compound A treatment at about 15 mg/day.

In one embodiment, the method as described herein provides a plasma Compound A AUC₈ₕ between about 10,000 ng*h/ml and about 14,800 ng*h/ml in the subject receiving Compound A treatment at about 25 mg/day. In one embodiment, the method as described herein provides a plasma Compound A AUC₈ₕ between about 9,840 ng*h/ml and about 14,760 ng*h/ml in the subject receiving Compound A treatment at about 25 mg/day. In one embodiment, the method as described herein provides a plasma Compound A AUC₈ₕ between about 11,100 ng*h/ml and about 13,500 ng*h/ml in the subject receiving Compound A treatment at about 25 mg/day.

In one embodiment, the method as described herein provides a plasma Compound A AUC₈ₕ between about 12,700 ng*h/ml and about 19,000 ng*h/ml in the subject receiving Compound A treatment at about 40 mg/day. In one embodiment, the method as described herein provides a plasma Compound A AUC₈ₕ between about 12,640 ng*h/ml and about 18,960 ng*h/ml in the subject receiving Compound A treatment at about 40 mg/day. In one embodiment, the method as described herein provides a plasma Compound A AUC₈ₕ between about 14,200 ng*h/ml and about 17,400 ng*h/ml in the subject receiving Compound A treatment at about 40 mg/day.

In one embodiment, the method as described herein provides a plasma Compound A AUC₈ₕ between about 30,000 ng*h/ml and about 45,000 ng*h/ml in the subject receiving Compound A treatment at about 60 mg/day. In one embodiment, the method as described herein provides a plasma Compound A AUC₈ₕ between about 33,800 ng*h/ml and about 41,300 ng*h/ml in the subject receiving Compound A treatment at about 60 mg/day.

In some embodiments, the AUC₈ₕ is measured in the subject's plasma. In some embodiments, the AUC₈ₕ is measured in the subject's blood. In some embodiments, the AUC₈ₕ is measured in the subject's plasma or blood on Cycle 2 Day 1. In some embodiments, the AUC₈ₕ is measured in the subject's plasma or blood on about Day 29 of treatment with Compound A.

In one embodiment, the subject receives the treatment provided herein for 1 to 12 cycles, wherein every cycle consists of about 28 days. In one embodiment, the subject receives the treatment provided herein for 1 to 12 cycles, wherein every cycle consists of about 21 days. In one embodiment, the subject receives the treatment provided herein for 1 to 12 cycles, wherein every cycle consists of about 14 days. In one embodiment, the subject receives the treatment provided herein for 1 to 12 cycles, wherein every cycle consists of about 7 days.

In one embodiment, the subject receives the treatment provided herein for 1 to 12 cycles, wherein every cycle consists of about 28 days. In one embodiment, the subject receives the treatment provided herein for 1 to 10 cycles, wherein every cycle consists of about 28 days. In one embodiment, the subject receives the treatment provided herein for 1 to 8 cycles, wherein every cycle consists of about 28 days. In one embodiment, the subject receives the treatment provided herein for 1 to 6 cycles, wherein every cycle consists of about 28 days. In one embodiment, the subject receives the treatment provided herein for 1 to 4 cycles, wherein every cycle consists of about 28 days. In one embodiment, the subject receives the treatment provided herein for 4 cycles, wherein every cycle consists of about 28 days. In one embodiment, the subject receives the treatment provided herein for 3 cycles, wherein every cycle consists of about 28 days. In one embodiment, the subject receives the treatment provided herein for 2 cycles, wherein every cycle consists of about 28 days. In one embodiment, the subject receives the treatment provided herein for 1 cycle, wherein every cycle consists of about 28 days.

In one embodiment, the subject achieves a stable disease, a partial response, or a complete response. In one embodiment, the subject achieves a partial response or a complete response. In one embodiment, the subject achieves a complete response. In one embodiment, the subject does not experience a progressive disease. In one embodiment, the subject achieves a stable disease. In one embodiment, the subject achieves a partial response. In one embodiment, the subject achieves a stable disease, a partial response, or a complete response for 1 week, 2 weeks, 3 weeks, or 4 weeks. In one embodiment, the subject achieves a stable disease, a partial response, or a complete response for 1 month, 2 months, 3 months, 4 months, 5 months, 6 months, 7 months, 8 months, 9 months, 10 months, 11 months, 12 months. In one embodiment, the subject achieves a stable disease, a partial response, or a complete response for 1 year, 2 years, 3 years, or 4 years.

Provided herein is a method of treating a cancer in a subject in need thereof, comprising administering to said subject an inhibitor of BRAF, wherein the cancer is characterized by a mutation selected from the group consisting of RAS, NRAS, KRAS, and their combination thereof; preferably NRAS, and KRAS, and their combination thereof; more preferably KRAS. Provided herein is a method of treating a cancer in a subject in need thereof, comprising administering to said subject an inhibitor of BRAF, wherein the cancer is characterized by a KRAS mutation.

In one embodiment, the cancer is selected from the group consisting of colorectal cancer, pancreatic cancer, melanoma, non-small cell lung cancer, brain cancer, lung cancer, kidney cancer, bone cancer, liver cancer, bladder cancer, breast, head and neck cancer, ovarian cancer, skin cancer, adrenal cancer, cervical cancer, lymphoma, thyroid tumor, and their complications; preferably melanoma, ovarian cancer, and non-small cell lung cancer; more preferably melanoma, and non-small cell lung cancer.

In one embodiment, the cancer is characterized by a mutation selected from the group consisting of NRAS Q61R, NRAS Q61K, NRAS Q61L, NRAS G12S, NRAS G13R, KRAS G12A, KRAS G12C, KRAS G12D, KRAS G12V, BRAF V600E, BRAF fusion, and their combination thereof; preferably NRAS Q61R, NRAS Q61K, NRAS Q61L, KRAS G12D, KRAS G12V, BRAF V600E, BRAF fusion, and their combination thereof; more preferably NRAS Q61R, NRAS Q61K, NRAS Q61L, KRAS G12D, KRAS G12V, and their combination thereof. In one embodiment, the cancer is characterized by a mutation provided herein.

### Methods for Making Compounds

The present embodiments can be understood more fully by reference to the detailed description and examples, which were intended to exemplify non-limiting embodiments.

Compound A is compound 1.49 in WO2014206343 and Compound 1 in WO2020151756. Compound A can be prepared as described in WO2014206343 and WO2020151756. In one embodiment, Compound A may be prepared according to the methods below:

### Step 1: Synthesis of INTQ-1

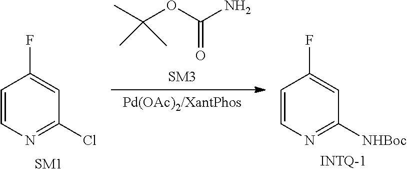

1,4-Dioxane (1.5 volumes) was added to 2 L 4-necked round-bottom flask and the flask was evacuated and flushed three times with nitrogen. Then Pd(OAc)₂ (2 wt %, 0.50 kg) and XantPhos (9 wt %, 2.25 kg) was add into the flash, and the flask was evacuated and flushed three times with nitrogen. The mixture was stirred at room temperature for 0.5˜1 hour under nitrogen atmosphere. NaOH (12.25 kg, 1.6 eq), H₂O (1 volumes, 25 L) and 1,4-dioxane (8 volumes, 200 L) were charged into a 20 L reactor. The mixture was stirred until clear, and then SM3 (26.75 kg, 1.2 eq) was added into the mixture. The solution of the catalyst was transferred to the above reactor under nitrogen atmosphere. Then SM1 (25.00 kg, 1.0 eq) was dropwise added into the reactor. The system was heated to 65±5° C. and maintained at 65±5° C. for at least 5 hrs. HPLC was used to monitor the reaction until the content of SM1 was not more than 1.0%. The reaction mixture was cooled to 30±5° C., and then filtered and the cake was washed with 1,4-dioxane (1.0 volumes). H₂O (4 volumes) was added into the filtrate and concentrated to 5 volumes. Then H₂O (2 volumes) was added into the residue and concentrated to 5 volumes. The residue was cooled to room temperature and filtered. The cake was washed with H₂O (2 volumes). Then the filter cake was slurried with IPA (2 volumes) at 25±5° C. for 3 hrs. The mixture was filtered and the filter cake was washed with IPA (0.5 volumes). The solid was dried in an oven under reduced pressure.

### Steps 2&3: Synthesis of INTQ-3

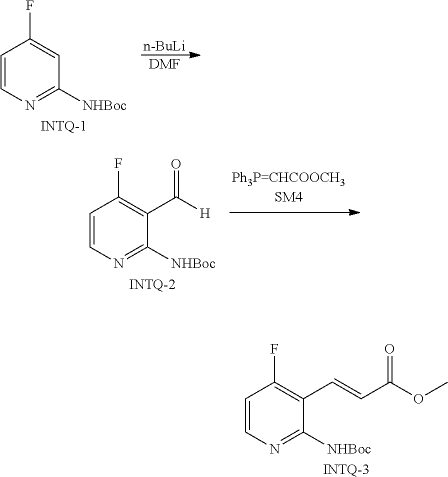

THF (25 volumes), and INTQ-1 (16.00 kg, 1.0 eq) were charged into the reactor. The mixture was stirred and cooled to −80˜−70° C. Then n-BuLi (n-hexane solution, 2.5M, 51.20 kg, 2.5 eq) was dropwise added into the mixture at −80˜−70° C. The reaction was monitored by TLC after reacting for 1-2 hrs at −80˜−70° C. Then DMF (9.92 kg, 1.8 eq) solution in THF (1.4 volumes) at −80˜−70° C. was dropwise added into the reaction system. The reaction was monitored by TLC after reacting for 1-2 hrs at −80˜−70° C. AcOH solution in THF (1.4 volumes) was dropwise added into the mixture to adjust the value of PH to 6-7 at −80˜−70° C. Then TEA (8.00 kg, 1.05 eq) was charged into the reaction at −80˜−70° C. The solution of methyl triphenylphosphoranylidene acetate (26.4 kg, 1.05 eq) in DCM (19 volumes) was dropwise added into the reaction mixture. The mixture was stirred for 10 hrs at −80˜−70° C., then the reaction was monitored by TLC. H₂O (10.5 volumes) and citric acid (32.00 kg, 2.1 eq) were charged into another reactor. The mixture was stirred to dissolve and cooled to 0-5° C. The temperature was cooled to −20° C. and the solution was transferred into the above 3 L 4-necked round-bottom flash. Then the mixture was stirred for 1 hour below 20° C. and the value of pH was confirmed to be between 4˜7. The organic layer was separated and washed with 25% NaCl (17 volumes). Then the organic phase was concentrated to 5 volumes and EtOAc (17 volumes) was charged into the mixture and concentrated to 5 volumes. EtOAc (17 volumes) was charged into the mixture and concentrated to 5 volumes. The solution was used for the next step directly.

### Step 4: Synthesis of INTQ-4

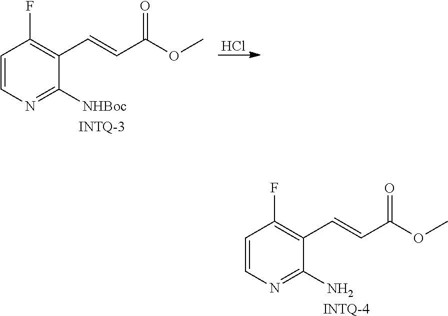

The solution of INTQ-3 in EtOAc was charged into a reactor. The solution was stirred and cooled to −5˜5° C. HCl was introduced into the mixture at −5˜5° C. for 2 hrs. Then the mixture was heated to 20˜30° C. HPLC was used to monitor the reaction every 2 hrs after reacting for 5 hrs until the content of INTQ-3 was less than 0.5%. The reaction mixture was concentrated to 10 volumes and cooled to 0˜5° C. The residue was stirred for 1 hour at 0˜5° C. The mixture was filtered and the filter cake was charged into H₂O (15 volumes). The mixture was stirred for 2 hrs at 20˜30° C. The mixture was filtered and the filter cake was washed with H₂O (3 volumes). Then the filtrate was transferred to another reactor and Na₂CO₃ was charged into the mixture to adjust the value of PH to 8˜9. Then the mixture was filtered and the filter cake was washed with H₂O (4 volumes). 20.73 kg (Yield: 69.0%, Purity: 95.0%) of INTQ-4 was given after drying in the vacuum oven.

### Step 5: Syntheses of INTQ-5

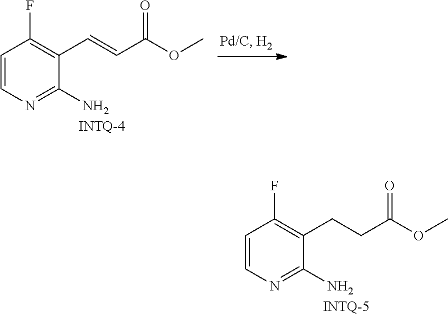

INTQ-4 (10.40 kg, 1.0 eq), Pd/C (15% wt, 1.25 kg) and THF (11 volumes) were charged into a reactor. The mixture was stirred and heated to 30˜35° C. Hydrogen was charged to a pressure of 10 atm. HPLC was used to monitor the reaction every 2 hrs after reacting for 15 hrs until the content of INTQ-4 was less than 0.5%. The reaction mixture was cooled to 20˜30° C. and filtered through Celite (0.2 wt). The filter cake was washed with THF (2 volumes). The filtrate was concentrated to 3 volumes and EtOH (6 volumes) was added into the mixture. The solution was concentrated to 3 volumes and EtOH (6 volumes) was charged into the mixture. The mixture was concentrated to 3 volumes and used for the next step directly.

### Step 6 Syntheses of BGB-INTQ-6

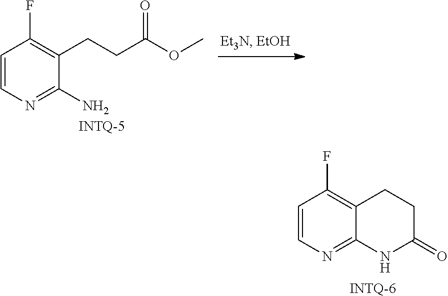

The solution of INTQ-5 (From the previous step) in EtOH (3 volumes), EtOH (7 volumes) and Et₃N (22% wt, 2.29 kg) were charged into the rector. The solution was heated to 70˜80° C. HPLC was used to monitor the reaction every 2 hrs after reacting for 15 hrs until the content of INTQ-5 is less than 1.0%. The reaction mixture was cooled to 30˜40° C. and concentrated to 5 volumes. The mixture was cooled to −5˜0° C. and stirred for 2 hrs. The mixture was filtered and the filter cake was washed with EtOH (1 volumes). 7.58 kg (Yield: 87.1%, Purity: 99.5%) of INTQ-6 was given after drying under oven at 45±5° C.

### Step 7: Synthesis of INTQ-7

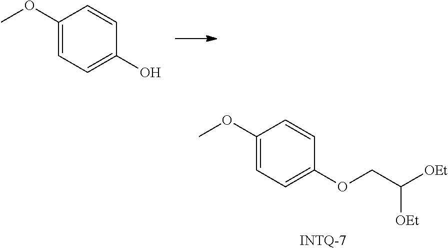

Potassium hydroxide (49.9 Kg, 1.7 equiv) was added to a solution of 4-methoxyphenol (65 Kg, 1.0 equiv) in DMSO (65 L, 1 volumes). The system was heated to 120° C. Bromoacetaldehyde diethyl acetal (123.8 Kg, 1.2 equiv) was dropwise added while maintaining the temperature at 120˜140° C. The reaction mixture was cooled to 20˜40° C. after reaction completion as monitored by HPLC. N-heptane (2 volumes) and water (2 volumes) was charged to the reaction mixture. The mixture was filtered through Celite (0.2 wt) and the filter cake was washed with n-heptane (0.5 volumes). The filtrate was stood for at least 30 minutes. The organic layer was separated and the aqueous layer was extracted with n-heptane (2 volumes). The combined organic layer was washed with 2 N aqueous NaOH (2 volumes). The organic layer was washed with 15% aqueous NaCl (2 volumes) two times. The organic layer was concentrated to 3 volumes. Toluene (3 volumes) was added and continued to concentrate to 3 volumes. The toluene solution of INTQ-7 was used for next step directly.

### Step 8: Synthesis of INTQ-8

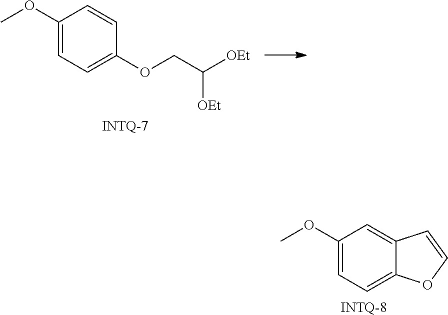

Amberlyst-15 (3.8 Kg, 0.1 wt) was added to toluene (760 L, 20 volumes). The system was heated to 110° C. under N₂ protection. The solution of INTQ-7 (38 Kg/Batch, 3 batches, 1.0 equiv) in toluene was dropwise added while maintaining the temperature at 105˜110° C. The reaction system was concentrated under constant pressure at 105˜110° C. to 17 volumes after reacting 1 hour. Toluene (3 volumes) was charged to the system. The reaction mixture was cooled to 20˜40° C. after reaction completion as monitored by HPLC. The mixture was filtered through Celite (0.1 wt) and the filter cake was washed with toluene (0.5 volumes). The filtrate was washed with 2 N aqueous NaOH (2 volumes). The organic layer was washed with 20% aqueous NaCl (2 volumes) two times. The organic layer was concentrated to 2 volumes. The crude product was distilled below 110° C. to given INTQ-8 as off-white solid (43 Kg, Yield=61.2%, Purity≥98.0%.

### Step 9: Synthesis of INTQ-9

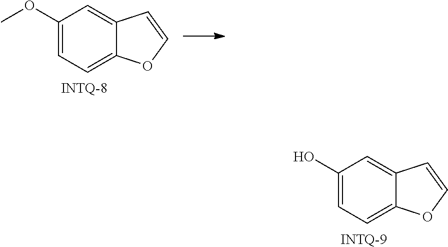

1-Dodecanethiol (147.0 Kg, 3.5 equiv) was added to a solution of INTQ-8 (43 Kg, 1.0 equiv) in NMP (260 L, 6 volumes). The system was heated to 75±5° C. Sodium ethoxide (69.0 Kg, 3.5 equiv) was added in portions while maintaining the temperature below 120° C. The reaction mixture was heated to 130±5° C. The mixture was sampled each hour for HPLC until the content of INTQ-8≤3.0% after reacting for 16 hours at 130±5° C. The reaction mixture was cooled to 60±5° C., and then 8 volumes water was charged to the mixture. The reaction mixture was cooled to 25±5° C., and then 3 volumes petroleum ether was charged to the mixture. The mixture was stirred for at least 30 minutes and stood for at least 30 minutes, separated. The organic phase was temporary storage. The aqueous phase was adjusted to pH=1˜2 with 6 N HCl. The aqueous phase was extracted with 5 volumes and 3 volumes ethyl acetate, respectively. The residue aqueous was combined with the temporary organic phase, and then 4 volumes ethanol and 4 volumes petroleum ether were charged. The mixture was stirred for at least 30 minutes and stood for at least 30 minutes, and then separated. The aqueous phase was adjusted to pH=1˜2 with 6 N HCl. The aqueous phase was extracted with 5 volumes ethyl acetate. The organic phase of ethyl acetate was combined and concentrated to 3 volumes under pressure below 50° C. 5 volumes n-heptane was charged to the residue and the mixture was adjusted to PH=9˜10 with 5% NaOH. The mixture was stirred for at least 30 minutes and stood for at least 30 minutes, separated. The aqueous phase was adjusted to pH=1˜2 with 6 N HCl. The aqueous phase was extracted with 5 volumes and 3 volumes ethyl acetate, respectively. Then the organic phases of ethyl acetate were combined and washed with 6 volumes 10% H₂O₂ and con. HCl (0.15 wt). Then the organic phase was washed with 6 volumes 5% H₂O₂ and con. HCl (0.15 wt). The organic layer was washed with 4 volumes 5% Na₂SO₃. The organic layer was washed with 3 volumes brine three times. The organic layer was concentrated to 3 volumes. Dichloromethane (5 volumes) was added and continued to concentrate to no obvious fraction. The crude product of INTQ-9 was used for next step directly.

### Step 10: Synthesis of INTQ-10

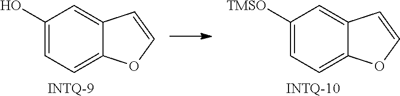

Et₃N (48.2 Kg, 2.0 equiv) was added to the solution of INTQ-9 (32 Kg, 1.0 equiv) in dichloromethane (10 volumes) below 40° C. The mixture was cooled to −5±5° C. TMSCI (1.3 equiv) in dichloromethane (1 volumes) was dropwise added while maintaining the temperature at −5±5° C. The mixture was sampled each hour for gas chromatography until the content of INTQ-9≤2.0% after reacting for 1 hour at '15±5° C. The mixture was concentrated to 3 volumes under pressure below 40° C. 15 volumes of n-hexane were charged to the residue and the mixture was stirred for at least 30 minutes. The mixture was filtered, and the filtrate was concentrated to no obvious fraction under pressure below 40° C. The crude product was distilled below 120° C. to given INTQ-10 as light-yellow oil (40 Kg, Yield=81.4%, Purity≥97.5%).

### Step 11: Synthesis of INTQ-11

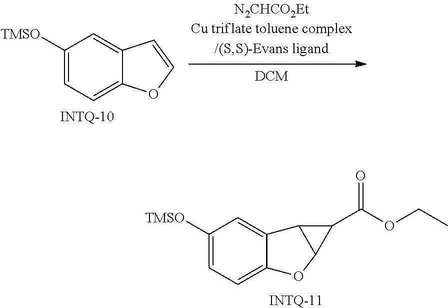

INTQ-10 (20 Kg/Batch, 2 batches, 1.0 eq) in dichloromethane (5 volumes) was slurried with CuI (0.1 wt) for 2˜3 hours at 25±5° C. Copper (I) triflate (2:1 complex with toluene, 0.11% wt) and (S,S)-2,2-Bis(4-phenyl-2-oxazolin-2-yl) propane (0.15% wt) were stirred in dichloromethane (4 volumes) at 20˜30° C. under N₂ atmosphere for 2˜3 hours. The solution of INTQ-10 in dichloromethane was added through microspores filter, the solution of ethyl diazoacetate (2.0 eq) in dichloromethane (10 volumes) was dropwise added slowly in 15˜25 hours at 20˜30° C. The mixture was stirred for 30˜60 minutes at 20˜30° C., the mixture was washed with 4 volumes 0.05N aqueous disodium edetate dihydrate three times at 20˜30° C. The organic section was washed with 3 volumes 25% aqueous NaCl two times. The organic section was concentrated under vacuum below 35° C. until the system was not more than 3 volumes. The crude product of INTQ-11 was used for next step directly.

### Steps 12 & 13: Synthesis of INTQ-13

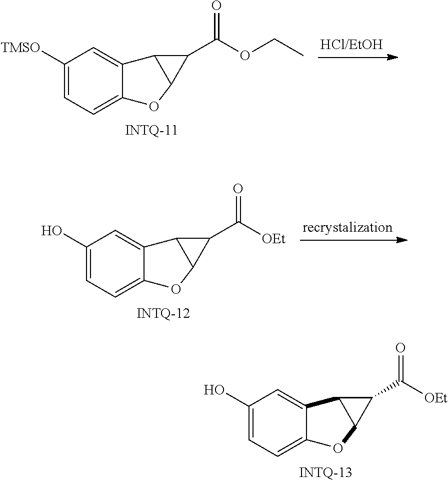

Step 12: The crude product of INTQ-11 was dissolved in methanol (3 volumes), 38% HCl in EtOH (0.1 volumes) was added into the mixture and stirred 2˜3 hours at 20˜30° C. Et₃N was dropwise added into the mixture to adjust PH=7. The mixture was concentrated under pressure to 2 volumes. Ethyl acetate (2 volumes) was charged and continued to concentrate under pressure to 2 volumes. N-heptane (2 volumes) was charged and continued to concentrate under pressure to 2 volumes. Dichloromethane (2 volumes) was charged for the material was dissolved completely. The residue was purified by silica gel chromatography (eluted with EtOAc: PE=1:5, about total 100 volumes) to give INTQ-12 as a yellow solid.

Step 13: INTQ-12 was charged to EtOAc (1.5 volumes) and n-heptane (20 volumes), the mixture was heated to 75˜85° C. until to clear. The clear solution was stirred for 1 hour at 75˜85° C. and then gradually cooled to 15˜20° C. The mixture was filtered and washed with n-heptane (2 volumes) to afford the product. The wet product was dried at 55±5° C. for at least 16 hours to give INTQ-13 as light yellow to off-white solid.

### Steps 14 & 15: Synthesis of INTQ-15

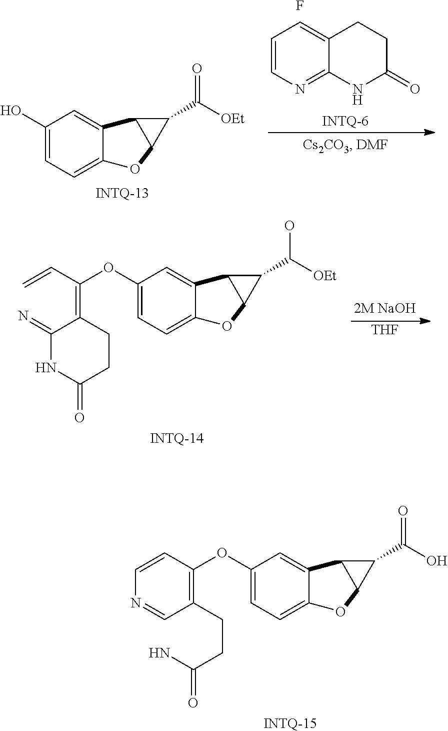

Step 14: INTQ-13 (16 Kg, 1.0 equiv) and INTQ-6 (12.7 Kg, 1.05 equiv) were added to DMF (5 volumes). The system was heated to 55±5° C. Cesium carbonate (29.6 Kg, 1.25 equiv) was added. The reaction mixture was heated to 110±5° C. The mixture was sampled each hour for HPLC until the content of INTQ-13≤0.5% after reacting for 2 hours at 110±5° C. The reaction mixture was cooled to 30±5° C., and then adjusted to pH-6 with acetic acid (5 wt) at 30±5° C. Water (30 volumes) was added to the mixture at 25±5° C. The mixture was stirred for 1˜2 hours and filtered to afford wet product. The wet product was re-slurry with water (5 volumes). The filter cake was used for next step directly.

Step 15: The wet product of INTQ-14 was added to mixture of 1 N NaOH (10 volumes) and THF (20 volumes). The system was stirred at 25±5° C. Sample each hour for HPLC until the content of INTQ-14≤0.5% after reacting for 4 hours at 25±5° C. The system was adjusted to pH=4˜5 with 4 N HCl at 25±5° C. and stirred for 1 hour. The system was concentrated to 8 volumes under pressure below 50° C. and then filtered to afford wet product. The wet product was re-slurry with THF (10 volumes). The mixture was stirred for 1˜2 hours and filtered to afford the wet product. The wet product was dried at 55±5° C. for at least 30 hours to give INTQ-15 as light brown to off-white solid.

### Steps 16&17&18: Synthesis of INTQ-18

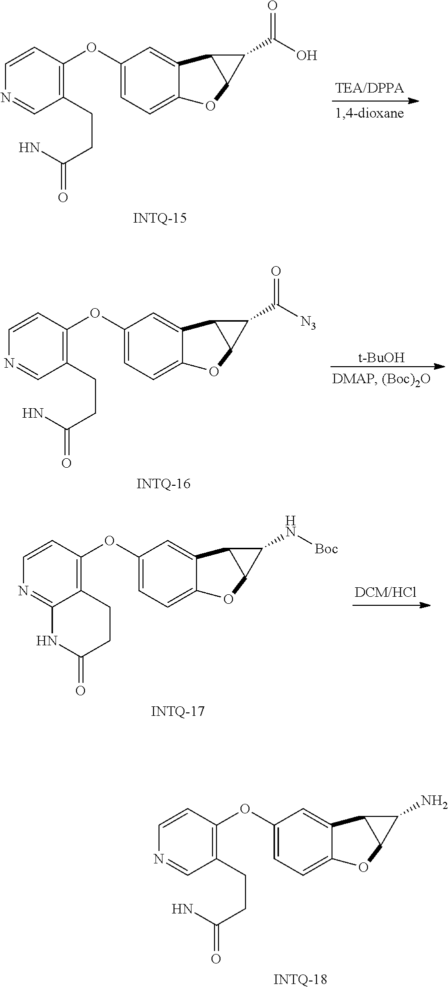

A reactor was vacuumed to ≤−0.08 MPa and then charged with inert nitrogen to atmosphere. 1,4-dioxane (10.0 volumes), INTQ-15 (3.6 kg, 1.0 eq) were added to the reactor. The mixture was concentrated to 6.0-6.5 volumes below 50° C. and the mixture was sampled for the content of water. Et3N (1.1 eq) was charged to the reactor. The mixture was heated to 30±5° C., and DPPA (1.1 eq) was dropwise added into the reactor. The mixture was sampled for HPLC analysis after reacting 2 hours at 30±5° C. until the content of INTQ-15≤1.0%. The solution of INTQ-16 was obtained.

Another reactor was vacuumed to ≤−0.08 MPa and then charged with inert nitrogen to atmosphere. t-BuOH (20.0 volumes), (Boc)₂O (0.5 eq), and DMAP (0.02 eq) were charged to the reactor. The mixture was heated to 85±5° C., stirred for at 2˜3 hours and the mixture was sampled for the content of water. The criterion is KF≤0.01%. The solution of INTQ-16 was dropwise added into the above reactor of t-BuOH system at 85±5° C. (duration of at least 3 hours). The mixture was sampled for HPLC analysis after 2 hours at 85±5° C. until the content of INTQ-16≤1.0%. The mixture was then cooled to less than 50° C., and concentrated to 3.0-4.0 volumes below 50° C.

DCM (10.0 volumes×2) was charged to the residue and the mixture was concentrated to 3.0-4.0 volumes below 50° C. DCM (10.0 volumes) was charged to the residue. Then 1 wt % NaOH aqueous (20.0 volumes) was charged to reactor and stir at 25±5° C. at least 1 hours. The mixture was filtered through Celite and then separated. The organic phase was washed with water (5.0 volumes) and separated. The organic phase was further washed with 25 wt % brine (5.0 volumes) and separated through silica gel to remove some impurities. The organic phase was concentrated to 6.0-7.0 volumes below 40° C. DCM was charged to 7.0 volumes. The mixture was then cooled to no more than 15° C., and hydrochloric acid (1.2 volumes) was added dropwise to the reactor at the temperature not more than 15° C. The mixture was sampled for HPLC analysis after reacted 3 hours at 15±5° C. until the content of INTQ-17≤4.0%. The mixture was heated to 25±5° C., water (3.0 volumes) was added to the reactor.

INTQ-16: ¹H NMR (400 MHz, DMSO-d6) δ 10.48 (s, 1H), 7.95 (d, J=5.6 Hz, 1H), 7.34 (d, J=2.4 Hz, 1H), 7.05 (d, J=8.4 Hz, 1H), 7.00 (dd, J=8.8, 2.4 Hz, 1H), 6.25 (d, J=5.6 Hz, 1H), 5.42 (d, J=5.2 Hz, 1H), 3.56 (dd, J=5.2, 2.8 Hz, 1H), 2.92 (t, J=7.6 Hz, 2H), 2.54 (d, J=8.0 Hz, 2H), 1.51 (d, J=3.2 Hz, 1H). MS: M/e 364 (M+1)⁺.

INTQ-17: ¹H NMR (400 MHz, DMSO-d6) δ 10.49 (s, 1H), 7.94 (d, J=6.0 Hz, 1H), 7.34 (s, 1H), 7.18 (s, 1H), 6.96-6.83 (m, 2H), 6.22 (d, J=5.6 Hz, 1H), 4.86 (d, J=5.6 Hz, 1H), 2.92 (t, J=7.6 Hz, 2H), 2.86 (d, J=4.8 Hz, 1H), 2.54 (t, J=7.6 Hz, 2H), 2.12 (s, 1H), 1.39 (s, 9H). MS: M/e 410 (M+1)⁺.

pH adjustment process: The solution of 4 wt % NaOH aq. was added dropwise into the reactor to adjust pH value to 2.7-3.1. If pH>3.1, hydrochloric acid (0.2 volume) was charged, then the solution of 4 wt % NaOH aq. was dropwise added into the reactor to adjust pH value to 2.7-3.1 (Precision pH test paper, range 2.7-4.7); the mixture was separate and the emulsion phase was collected as aqueous phase. The mixture was filtered through Celite, and the resulting aqueous phase was washed with DCM (2.0 volumes) once. Into the remaining aqueous phase in the reactor, DCM (6.0 volumes) and EtOH (5.0 volumes) were charged. 10.0 wt % Na₂CO₃ solution was added dropwise into the reaction to adjust the value of pH to 8-9 at 25±5° C. The mixture was stirred for 10-15 min and stood for 10-15 min. The mixture was separated, and the aqueous phase was extracted with DCM (4.0 volumes) for 2 times. The organic phase was combined and washed with water (2.0 volumes), separated and the organic phase was washed with 25 wt % brine (5.0 volumes) once. The organic phase was concentrated to 3.0-4.0 volumes below 45° C., then n-heptane (4.0 volumes) was charged to the residual. The mixture was concentrated to 3.0-4.0 volumes below 45° C., and then n-heptane (4.0 volumes) was charged to the residual. The mixture was concentrated to 3.0-4.0 volumes below 45° C. The residual was cooled to 25±5° C., and then centrifuged and the solid was washed with n-heptane (2.0 volumes). The cake was transferred to a vacuum oven and the mixture was sampled for Loss on Drying (LOD) until LOD≤1.0% after dry for 4 hours at 45±5° C. (box temperature). The purity of INTQ-18 (2.25 kg) was reported. The product was packaged in double LDPE plastic bags and stored at 2-30° C.

INTQ-18: ¹H NMR (400 MHz, DMSO-d6) δ 10.81 (s, 1H), 8.87 (s, 3H), 8.05 (d, J=6.0 Hz, 1H), 7.33 (t, J=1.2 Hz, 1H), 7.07-6.95 (m, 2H), 6.34 (d, J=6.0 Hz, 1H), 5.24 (d, J=6.0 Hz, 1H), 3.32 (dd, J=6.0, 2.0 Hz, 1H), 2.97 (t, J=7.6 Hz, 2H), 2.59 (t, J=7.6 Hz, 2H), 2.46 (s, 1H). MS: M/e 310 (M+1)⁺.

### Step 19: Synthesis of INTQ-19

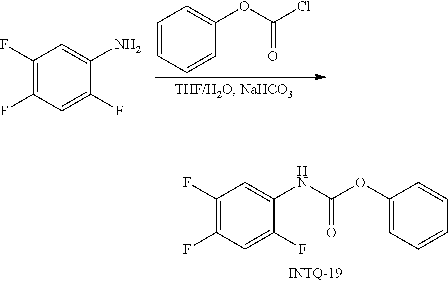

The reactor was vacuumed to ≤−0.08 MPa and then charged with inert nitrogen to atmosphere. THF (6.0 volumes), H₂O (3.0 volumes), 2, 4, 5-trifluoroaniline (1.0 eq), NaHCO₃ (1.2eq) were charged to the reactor. The mixture was cooled to 0° C., phenyl chloroformate was added slowly at 0±5° C. The mixture was stirred for at least 2 hours. The mixture was sampled for LCMS until 2, 4, 5-trifluoroaniline≤0.2%. EA (15.0 volumes) was then added. The organic phase was washed with H₂O (5.0 volumes), and then washed with 5wt % HCl aq. (5.0 volumes) for 2 times, washed with Sat. NaCl (5.0 volumes) for 2 times. The organic phase was concentrated to 10.0 volumes below 45° C. N-heptane (10.0 volumes) was charged to the residual. The mixture was concentrated to 10.0 volumes, and then n-heptane (10.0 volumes) was charged to the residual. The mixture was concentrated to 10.0 volumes and centrifuged and the solid was washed with n-heptane (2.0 volumes). The cake was sampled for LCMS analysis with the criterion of INTQ-19>99%. The cake was then transferred to a vacuum oven and sampled for LOD until LOD≤2.0% after dry for 10 hours at 35±5° C. (box temperature). The purity of INTQ-19 was reported. The product was packaged in double LDPE plastic bags and stored at 2-30° C.

INTQ-19: ¹H NMR (400 MHz, DMSO-d6) δ 10.20 (s, 1H), 7.82 (dt, J=12.0, 8.0 Hz, 1H), 7.66 (td, J=10.8, 7.6 Hz, 1H), 7.44 (t, J=7.6 Hz, 2H), 7.33-7.20 (m, 3H).

### Step 20: Syntheses of Crystalline Form of Compound A (Form A)

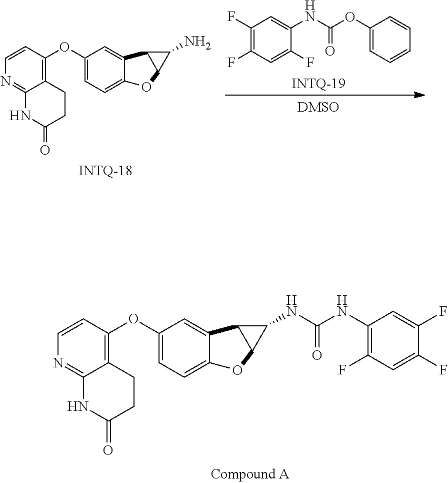

The reactor was vacuumed to ≤−0.08 MPa and then charged with inert nitrogen to atmosphere. DMSO (9.0 volumes), INTQ-18 (1.63 kg, 1.0eq) and N-methyl morpholine (1.0 eq) were charged to the reactor. The mixture was stirred for at least 0.5 hour at 20±5° C. INTQ-19 (1.27 kg, 0.9 eq) was charged to the reactor at 20±5° C. The mixture was sampled for HPLC analysis after reacting for 3 hours at 20±5° C. until the content of INTQ-19≤0.3%. After the completion of the reaction, the mixture of Compound 1 was dropwise added through microfilter into solution of 0.5% hydrochloric acid which was also filtered through a micron filter (30.0 volumes) slowly at 20±5° C. The mixture was stirred for at least 4 hours, and centrifuged. The filter cake was washed with purified water (5.0 volumes×2).

Slurry procedure: DMSO (9.0 volumes) and 0.5% hydrochloric acid were charged through a micron filter (30.0 volumes) to a reactor, and the filter cake was charged to the reactor and the mixture was stirred for at least 4 hours at 20±5° C., and then centrifuged. The filter cake was washed with purified water (5.0 volumes×2). The cake was sampled for HPLC analysis with the criterion of Compound 1≥98.0% If Compound 1<98.0%, “Slurry procedure” is repeated. Purified water (40.0 volumes) and filter cake were charged to a reactor, and the mixture was stirred for at least 4 hours at 20±5° C., and then centrifuged. The filter cake was washed with purified water (5.0 volumes×2). The cake was then dried under vacuum at 45±5° C. for at least 8 hours until LOD≤3.0%. If the solvent residue cannot meet the criteria, removal of residual solvent by slurry: purified water (40.0 volumes) and product were charged to a reactor, and the mixture was stirred for at least 4 hours at 20±5° C., and then centrifuged. The filter cake was washed with purified water (5.0 volumes×2). The cake was dried under vacuum at 45±5° C. for at least 8 hours until LOD≤3.0%. The cakes were sampled for solvent residue. If solvent residue cannot meet criteria, the procedure “removal of residual solvent by slurry” is repeated until solvent residue meets the criterion. The material was sampled for HPLC analysis with the criterion of Compound 1≥98.0% purity (2.02 kg) and the criterion of impurity-1 less than 0.5%. HPLC analysis determined that the content of Impurity-1 was less than 0.1% herein. The product was packaged in double LDPE bags with desiccant, stored at room temperature.

Compound A: ¹H NMR (400 MHz, DMSO-d6) δ 10.47 (s, 1H), 8.54 (s, 1H), 8.23-8.07 (m, 1H), 7.96 (d, J=5.6 Hz, 1H), 7.65-7.51 (m, 1H), 7.23 (s, 1H), 7.01 (d, J=2.0 Hz, 1H), 6.96-6.87 (m, 2H), 6.25 (d, J=5.6 Hz, 1H), 4.98 (d, J=6.0 Hz, 1H), 2.97 (dd, J=5.6, 1.6 Hz, 1H), 2.93 (t, J=7.6 Hz, 2H), 2.54 (t, J=7.6 Hz, 2H), 2.26 (s, 1H).

The present embodiments can be understood more fully by reference to the detailed description and examples, which are intended to exemplify non-limiting embodiments.

### EXAMPLES

The examples below were intended to be purely exemplary and should not be considered to be limiting in any way. Unless otherwise specified, the experimental methods in the Examples described below were conventional methods.

TABLE 1
List of Abbreviations and Terms

| Abbreviation | Definition |
|---|---|
| AE | adverse event |
| A-RAF | A-RAF proto-oncogene |
| AUC | area under the plasma concentration time curve |
| AUC_{0-∞} | area under the plasma concentration time curve from zero |
| | to infinity |
| AUCₗₐₛₜ | area under the plasma concentration time curve from zero |
| | to the last measurable concentration |
| BID | twice daily |
| B-RAF | v-RAF murine sarcoma viral oncogene homolog B |
| C-RAF | Raf-1 proto-oncogene serine/threonine kinase |
| CI | confidence interval |
| Cmax | maximum observed plasma concentration |
| CR | complete response |
| CRC | colorectal cancer |
| CT | computed tomography |
| CTCAE | Common Terminology Criteria for Adverse Events |
| CYP | cytochrome P450 |
| DLT | dose-limiting toxicity |
| ECG | electrocardiogram |
| ECHO | echocardiogram |
| ECOG | Eastern Cooperative Oncology Group |
| eCRF | electronic case report form |
| EDC | electronic data capture (system) |
| EGFR | epidermal growth factor receptor |
| ERK | extracellular signal-regulated kinase |
| FDG-PET | fluorodeoxyglucose PET |
| HBsAg | hepatitis B surface antigen |
| HCC | hepatocellular carcinoma |
| IB | Investigator's Brochure |
| IC₅₀ | concentration of an inhibitor where the response (or |
| | binding) was reduced by half |
| ICF | informed consent form |
| ICH | International Council for Harmonisation |
| IEC | Independent Ethics Committee |
| IRB | Institutional Review Board |
| K-RAS | Kirsten rat sarcoma virus oncogene |
| LVEF | left ventricular ejection fraction |
| MAPK | mitogen activated protein kinase |
| MedDRA | Medical Dictionary for Regulatory Activities |
| MEK | MAPK/ERK kinase |
| MRI | magnetic resonance imaging |
| MTD | maximum tolerated dose |
| NF1 | neurofibromin 1 |
| N-RAS | neuroblastoma RAS viral oncogene homolog |
| NSCLC | non-small cell lung carcinoma |
| ORR | objective response rate |
| PD | pharmacodynamics |
| PET | positron emission tomography |
| PFS | progression-free survival |
| PK | pharmacokinetic(s) |
| PR | partial response |
| QD | once a day |
| RAF | rapidly accelerated fibrosarcoma |
| RECIST | Response Evaluation Criteria in Solid Tumors |
| RP2D | recommended phase 2 dose |
| RVO | retinal vein occlusion |
| SAE | serious adverse event |
| SMC | Safety Monitoring Committee |
| SS | steady state |
| T_{1/2} | elimination half-life |
| TEAE | treatment-emergent adverse event |
| Tₘₐₓ | time to maximum observed plasma concentration |
| ULN | upper limit of normal |

### Example 1

The entire disclosure of NCT04249843 on ClinicalTrials.gov was incorporated herein by reference.

The study was a multicenter, open-label, 2-part (dose-escalation and expansion) Phase 1 study of Compound A in patients with tumors harboring B-RAF or K-RAS/N-RAS mutations that may respond to a RAF dimer inhibitor.

### Objectives and Endpoints

### Study Objectives for Dose Escalation Part 1a

### Primary Objectives

To assess the safety and tolerability of Compound A in patients with solid tumors.

To determine the MTD, if any, and recommended Phase 2 dose (RP2D) for Compound A.

### Secondary Objectives

To characterize the pharmacokinetics of Compound A after single-dose and multiple-dose administration.

To assess the preliminary antitumor activity of Compound A.

### Exploratory Objectives

To determine potential predictive biomarkers of efficacy.

To assess potential pharmacodynamic biomarkers of target engagement, biological activity, and mechanism of action.

To explore mechanisms of treatment resistance in patients who fail to respond or develop resistance.

### Study Objectives for Dose Expansion Part 1b

### Primary Objectives

To determine the objective response rate (ORR) (confirmed complete response [CR] or partial response [PR]) as assessed by Response Evaluation Criteria in Solid Tumors (RECIST version [v] 1.1) of Compound A at the RP2D when given orally in patients with selected tumors.

### Secondary Objectives

To determine the progression-free survival (PFS); disease control rate (DCR; confirmed CR, PR, or stable disease [SD]) as assessed by RECIST v1.1; duration of response (DOR); and overall survival of Compound A as a single agent.

To further assess the safety and tolerability of Compound A.

To further characterize the pharmacokinetics of Compound A.

### Exploratory Objectives

To determine potential predictive biomarkers of efficacy.

To assess potential pharmacodynamic biomarkers of target engagement, biological activity and mechanism of action.

To explore mechanisms of resistance in patients who fail to respond or develop resistance.

### Study Endpoints for Dose Escalation Phase 1a

### Primary Endpoints

Safety and tolerability of Compound A were assessed by the incidence of serious adverse events (SAEs) and the incidence and severity of adverse events (AEs) (coded to preferred term [PT] and system organ class [SOC] using the Medical Dictionary for Regulatory Activities [MedDRA]), with dose-limiting toxicities (DLTs) and AEs graded according to the National Cancer Institute Common Terminology Criteria for Adverse Events version 5.0 (NCI-CTCAE v5.0), physical examination, vital signs, electrocardiograms (ECGs), and laboratory tests.

The MTD were determined by a Safety Monitoring Committee (SMC) based on the occurrence of DLTs, safety, and tolerability. The SMC defined the RP2D based on safety, preliminary efficacy, and other complementary data.

### Secondary Endpoints

For a single-dose profile: AUC from zero to the last measurable concentration (AUClast), AUC from zero to infinity (AUC0-∞), maximum observed plasma concentration (Cmax), time to maximum observed plasma concentration (tmax), t1/2, apparent clearance, and apparent volume of distribution (Vz/F). In addition, AUClast,ss, AUCtau, Cmax,ss, and tmax, ss were calculated at steady state.

Objective response rate was defined as the proportion of patients who had confirmed CR or PR, assessed by the investigator using RECIST v1.1.

Duration of response was defined as the time from the first determination of an objective response per RECIST v1.1 until the first documentation of progression or death due to any cause, whichever occurs first.

Clinical benefit rate was defined as the proportion of patients with best overall response (BOR) of confirmed CR, PR, or SD lasting ≥16 weeks.

Progression-free survival was defined as the time from the date of the first dose of investigational medicinal product (IMP) to the first documented disease progression per RECIST v1.1 or death due to any cause, whichever occurs first.

Duration of SD (DSD) was defined as the time interval, in the absence of either confirmed CR or PR, between the date of the first dose of IMP and the first documented disease progression per RECIST v1.1 or death due to any cause, whichever occurs first.

### Exploratory Endpoints

Predictive biomarkers of efficacy, including but not limited to MAPK signaling including pERK level, B-RAF and K-RAS mutational status, and other aberrations in the MAPK pathway.

Identification of potential biomarkers of resistance: optional tumor biopsy samples taken at disease progression were used to identify potential biomarkers of resistance, including but not limited to expression (protein and RNA) of MAPK signaling.

### Study Endpoints for Dose Expansion Phase 1b

### Primary Endpoint

Objective response rate (confirmed CR+PR) as described in Section titled “Secondary Endpoints” above in patients with selected tumors.

### Secondary Endpoints

Progression-free survival as described above, DCR (confirmed CR, PR, or SD), DOR, CBR (confirmed CR or PR or durable SD [SD≥16 weeks]), and DSD.

Overall survival was defined as the time from the date of the first dose of IMP to the date of death due to any cause.

Safety and tolerability assessment of AEs, SAEs, physical examination, ECGs, and laboratory measurements.

Single dose and steady state pharmacokinetics, including but not limited to Ctrough.

### Exploratory Endpoints

As described for Phase 1a as provided above.

### Study Design

### Summary of Study Design

This was a 2-part Phase 1 study of Compound A in patients with tumors harboring B-RAF mutations that were likely to respond to a RAF dimer inhibitor. Compound A was a second-generation B-RAF inhibitor that has demonstrated potent inhibitory activity against the RAF family of serine/threonine kinases. In preclinical studies, Compound A showed it inhibited tumor cell lines harboring non-V600 B-RAF mutations; it was also active towards B-RAF/MEK inhibitor-resistant tumors.

Phase 1a consisted of a dose-escalation and dose-finding component to establish the MTD and/or RP2D and to evaluate the pharmacokinetics of Compound A. Phase 1b consisted of an expansion component to further evaluate the pharmacokinetics, safety, and tolerability of Compound A at the RP2D and to assess the preliminary antitumor activity of the compound in patients in each of 2 treatment groups.

Baseline tumor tissue was mandatory for mutation and biomarker analysis, either from archived tumor tissue or fresh tumor biopsies. For patients with readily accessible tumor lesions who consent to the biopsies, a follow-up biopsy may be taken, ideally from the same tumor lesion, for the analysis of pharmacodynamic biomarkers. For Group 2 in Phase 1b, paired fresh tumor biopsies within 8 weeks prior to first dose of IMP and at a time post-dose to assess PD biomarkers were mandatory. In addition, blood samples were collected from all patients for mutation and biomarker analysis.

### Phase 1a

Dose escalation occurred in accordance with the modified toxicity probability interval (mTPI-2) mTPI-2 design (Yuan Ji et al 2010).

The mTPI-2 uses a Bayesian statistics framework and a beta/binomial hierarchical model to compute the posterior probability of 3 dosing intervals that reflect the relative difference between the toxicity rate of each dose level. The target toxicity rate for the MTD was ϕ=0.33. The maximum sample size was 30. The trial enrolled and treated patients in minimum cohorts of 3 patients. Being a model-based design, mTPI-2 automatically and appropriately tailors dose-escalation and de-escalation decisions with the acceptable toxicity probability interval of (0.28, 0.405). All the dose-escalation decisions were pre-calculated under the mTPI-2 design and presented in a two-way table. The decision rules to “dose escalate” (E), “no change in dose” (S), “dose de-escalate” (D) or “dose de-escalate, unacceptable toxicity” (DU) were described in Table 2.

Cohorts of patients could receive doses already tested but a dose that was associated with decision “Dose de-escalate, unacceptable toxicity” cannot be revisited and no additional patients should be treated at this dose or higher doses for the remainder of the trial.

TABLE 2
Dose Escalation/de-escalation Rule for the mTPI-2 Design

| Number | | | | | | | | | | | | | | | |
|---|---|---|---|---|---|---|---|---|---|---|---|---|---|---|---|
| of DLTs | n = 1 | n = 2 | n = 3 | n = 4 | n = 5 | n = 6 | n = 7 | n = 8 | n = 9 | n = 10 | n = 11 | n = 12 | n = 13 | n = 14 | n = 15 |
| 0 | E | E | E | E | E | E | E | E | E | E | E | E | E | E | E |
| 1 | D | D | S | E | E | E | E | E | E | E | E | E | E | E | E |
| 2 | | DU | D | D | S | S | S | E | E | E | E | E | E | E | E |
| 3 | | | DU | DU | D | D | D | S | S | S | E | E | E | E | E |
| 4 | | | | DU | DU | DU | D | D | D | S | S | S | S | S | E |
| 5 | | | | | DU | DU | DU | DU | D | D | D | D | S | S | S |
| 6 | | | | | | DU | DU | DU | DU | DU | D | D | D | D | S |
| 7 | | | | | | | DU | DU | DU | DU | DU | DU | D | D | D |
| 8 | | | | | | | | DU | DU | DU | DU | DU | DU | DU | DU |

Sample size/dose level=15, Target toxicity probability=0.33, epsilon 1=0.05, epsilon 2=0.075
Column indicates the number of patients treated. Row indicates the number of patients with DLTs
E: Escalate to the next higher dose; S: Stay at the same dose; D: De-escalate to the previous lower dose; DU: De-escalate to the previous lower dose and the current dose was never used again in the trial.

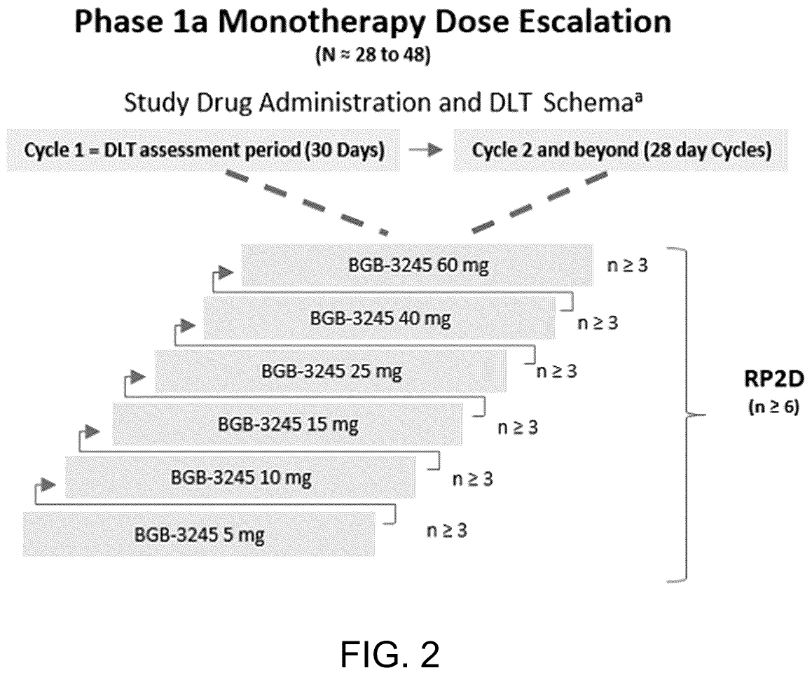

The study enrolled and treated patients in minimum cohorts of 3 patients (see FIG. 2). The design was described as follows:

Patients in the first cohort were treated at dose level 1.

To assign a dose to the next cohort of patients, conduct dose escalation/de-escalation according to Table 2. When using Table 2, please note the following:

“DU” means the current and higher doses from the trial were eliminated to prevent treating any future patients at these doses.

When a dose was eliminated, de-escalate the dose to the next lower level. When the lowest dose was eliminated, stop the trial for safety. In this case, no dose should be selected as the MTD.

If the current dose was the lowest dose and the rule indicates dose de-escalation (D), continue to treat the new patients at the lowest dose unless the number of DLTs reaches the elimination boundary (i.e., DU), at which point terminate the trial for safety.

If the current dose was the highest dose, which may be higher than the predefined dose level of 60 mg, and the rule indicates dose escalation, treat the new patients at the highest dose (or the dose that was recommended by SMC).

Repeat step 2 until the maximum sample size of 30 was reached or stop the trial if the number of patients treated at the current dose reaches 15.

Due to binomial data variability in small samples, DLTs may be observed in lower dose levels by chance even when the true Probability (DLT) was fairly low. This could result in the estimated posterior DLT rate to exceed the targeted very early in the trial, triggering an early stop when very few patients (e.g., 3) have been treated. To prevent stopping the trial prematurely in such cases, a dose de-escalation option with a lower dose of 5 mg or current dose level could be added to the dose grid.

Estimating the MTD and RP2D:

An MTD may not be identified, as DLTs may not be frequently observed after administration of this investigational agent. The study continued accruing until one of the 2 stopping conditions below was triggered.
1. MTD and/or RP2D has been identified with sufficient accuracy: at least 6 to 12 patients have been accumulated on a dose that was currently estimated to be the MTD and/or RP2D; or
2. All doses explored appear to be overly toxic.

The starting dose were 5 mg/day (5 mg QD). The 30-day initial treatment cycle (Cycle 1) of each dose level cohort consisted of a single administration of Compound A on Day 1, followed by a 2-day treatment-free period (Days 2 and 3) and a 27-day period of repeated drug administration (Days 4 to 30).

Continuous safety evaluation was performed by the SMC, which consists of the sponsor, the coordinating investigator, and the investigators. During dose escalation, the SMC made decisions on dose levels and/or adjustments to the dose regimen (i.e., dose level and/or schedule) based upon available data from the current as well previous dose levels. A minimum of 3 DLT evaluable patients for that dose level was required prior to determining the dose level and dose regimen for the next cohort. The RP2D and dosing regimen to be used in Phase 1b were recommended by the SMC and determined by the sponsor based on the available safety, pharmacokinetics, preliminary antitumor activity, and other complementary data from Phase 1a. The Sponsor may evaluate dose levels and/or schedules that have not been predefined or eliminated as long as it does not exceed 60 mg/day or the MTD level (whichever was less).

### Phase 1b

Phase 1b was a multicenter, open-label, 2-group, dose expansion study. Phase 1b of the study investigated the RP2D to examine the potential efficacy as well as safety and tolerability of Compound A in various advanced solid tumor indications. There were 2 treatment groups:
Group 1: solid tumors with non-V600 B-RAF mutations including RAF fusions, approximately 15 patients; and
Group 2: B-RAF V600 mutated melanoma or NSCLC that has progressed on a B-RAF-inhibitor and/or MEK-inhibitor, approximately 15 patients.

### Phase 1a and Phase 1b

Patients were monitored for safety, tolerability, and efficacy throughout the study from the day of first administration of IMP up to 30 (+7) days after the last administration of the IMP.

Patients initially enrolled into the Phase 1a (dose escalation component of the study). After completion of Phase 1a and once the RP2D for Compound A monotherapy has been determined, patients may be enrolled to different groups of the Phase 1b (advanced solid tumors) in parallel.

Tumor response was assessed by investigators based on RECIST v1.1. For patients with ovarian cancer, tumor response was also assessed by investigators based on Gynecological Cancer Intergroup (GCIG) CA-125 criteria. The RECIST criteria took priority regarding patient treatment and discontinuation decisions.

Patients in both phases of the study who continue to demonstrate clinical benefit at the end of the 2-year treatment were automatically rolled over to a new study protocol to continue receiving treatment and follow-up for safety and other clinically relevant information.

### Study Periods, Visits, Follow-Up, or Discontinuation

This study consists of the following 3 periods.

### Screening Period

Screening evaluations were performed within 21 days prior to the first administration of IMP. Patients who agree to participate signed the informed consent form (ICF) prior to undergoing any Screening procedure. Screening evaluations may be repeated as needed within the Screening Period; however, screening tumor assessments should not be repeated unless clinically indicated. The investigator was to assess patient eligibility according to the latest Screening assessment results.

Rescreening under limited conditions may be allowed after consultation with the medical monitor or designee (e.g., when a patient's laboratory result narrowly miss laboratory criterion and it was correctable and not due to rapidly deteriorating condition or PD). Rescreening was allowed only once. If a subject was re-screened, a new ICF were required.

Baseline tumor tissue was mandatory for mutation and biomarker analysis, either from archived tumor tissue or fresh tumor biopsies.

### Enrollment

All screening results and relevant medical history must be available before eligibility can be determined. The investigator must confirm that all eligibility criteria were met. The study site personnel completed the Eligibility Authorization Packet, which must be signed by the investigator or a sub-investigator. The medical monitor or designee reviewed and approved, if appropriate. Approval of the Eligibility Authorization Packet and corresponding date of approval marks the patient's date of enrollment. No eligibility waivers were granted. The study site personnel must ensure that confirmation of eligibility by the medical monitor has been received before the patient receives the first dose of study drug(s).

### Treatment Period

After completing all Screening activities, patients confirmed eligible and enrolled in the study were treated with Compound A.

The start of treatment (i.e., Cycle 1 Day 1) with Compound A must begin within 5 days of a patient's enrollment.

Cycle 1 Day 1 was the first day of study treatment. For Phase 1a, the first treatment Cycle 1 were 30 days, with a 2-day rest period on Days 2 and 3. Starting with Cycle 2 in Phase 1a, patients were treated by repeated treatment cycles of treatment cycles were 28 days of continuous treatment (i.e., no rest period). All patients received IMP until 1) disease progression; 2) cessation of study treatment due to death, intolerance, or withdrawal of consent from the study; or 3) completion of 2 years of treatment they meet one of the discontinuation criteria were met. Patients in both phases of the study who continue to demonstrate clinical benefit at the end of the 2-year treatment may continue to receive therapy per protocol or may enter an expanded access protocol or compassionate use assuming drug availability.

Study procedures of each clinic visit were outlined.

### Unscheduled Visits

Unscheduled visits may occur any time as necessary as per investigator decision or patient's request for reasons such as additional assessment or follow-up of AEs. If PD was suspected, a tumor assessment should be performed.

### Permanent Discontinuation of Study Treatment

Patients may permanently discontinue study treatment for any of the following reasons:
Disease progression
Completed 2 years of treatment
Adverse event(s)
Death
Pregnancy
Major protocol deviations
Patient withdrew consent for study treatment
Start of other anticancer therapy
Every effort must be made to encourage the patient to complete their end of treatment (EOT) visit and appropriate safety follow-ups.
Patient Discontinuation From the Study (End of Study for an Individual Patient)

Patients may discontinue from the study for reasons that include, but were not limited to, the following:
Patient withdrawal of consent
Death
Lost to follow-up
Completion of all study assessments
End-of-Treatment Visit

All patients underwent an EOT visit within 7 days after stopping all study treatments.

A visit was scheduled as soon as possible, but the EOT visit may occur later under specific circumstances (e.g., hospitalization) and after discussion with the medical monitor. The visit at which tumor assessment showed PD may be used as the EOT visit provided that all required assessments were performed. Tumor assessment does not have to be repeated if it was performed within 14 days of the EOT visit or at a prior response evaluation that documented PD. Electrocardiogram does not have to be repeated if it was performed within 14 days of the EOT visit.

### Safety Follow-Up Period

Patients returned for a follow-up visit at approximately 30 (+7) days after last dose of the IMP. Patients who discontinue IMP due to a drug-related AE were followed until the resolution of the AE (to Grade 1, baseline, or stabilization) or initiation of a new treatment, whichever comes first. If new anticancer therapy was inadvertently initiated before this safety follow-up (e.g., without the knowledge of the study center team), a safety follow-up should be scheduled as soon as possible.

### Lost to Follow-Up

If attempts to contact the patient by phone were unsuccessful, the following additional attempts should be made to obtain protocol-required follow-up information. The patient should be contacted by mail in a manner that provides proof of receipt by the patient. If unsuccessful, other contacts should be explored, such as referring physicians or relatives. Attempts of contact should be documented in the patient's source documents. If a patient cannot be contacted despite all attempts, the patient were considered lost to follow-up, and death information should be obtained through a public record search if local agencies permit.

### End of Study

The end of study was defined as the timepoint when the final data for a clinical study were collected, which was after final visit/follow up has occurred for the last study patient.

The sponsor has the right to terminate this study at any time. Reasons for terminating the study early include but were not limited to the following:
The incidence or severity of AEs in this or other studies indicates a potential health hazard to patients
Overall patient enrollment was unsatisfactory

The sponsor notified each investigator if a decision was made to terminate the study. Should this be necessary, prematurely discontinued patients must be seen for an EOT Visit and Safety Follow-up Visit.

The investigators may be informed of additional procedures to be followed to ensure that adequate consideration was given to the protection of the patients' interests. The investigator was responsible for informing IRBs/IECs of the early termination of the study.

The sponsor has the right to close a site at any time. The decision was communicated to the site in advance. Reasons for closing a site include but were not limited to the following:
Excessively slow recruitment
Poor protocol adherence
Inaccurate or incomplete data recording
Noncompliance with GCP or applicable laws and regulations
Study activity was complete (i.e., all study-related activities have been completed and all obligations have been fulfilled)
Dose-Limiting Toxicity Definition

All DLTs in Phase 1a were graded. The occurrence of any of the following toxicities within 30 days following the first dose of Compound A, if judged by the investigator as related to Compound A, were considered a DLT.

### Hematologic:

Any Grade 4 or above toxicity.

Grade 3 febrile neutropenia (defined as absolute neutrophil count (ANC) <1000/mm3 with a single temperature of >38.30° C. (101° F.) or a sustained temperature of ≥38° C. (100.4° F.) for >1 hour).

Grade 3 neutropenia with infection.

Grade 3 thrombocytopenia with clinically significant bleeding.

### Nonhematologic:

Any Grade 4 or above unless otherwise noted below.

Grade 3 toxicity that was clinically significant and does not resolve to baseline or ≤Grade 1 within 3 days of initiating optimal supportive care.

Grade ≥3 total bilirubin or hepatic transaminases (ALT or AST)

Note: The following AEs were not considered as DLTs:

Grade 3 rash.

Grade 3 or Grade 4 laboratory abnormalities that were not clinically significant or associated with clinical sequelae and were reversible within 48 hours

In addition, clinically important or persistent toxicities that were not included above may also be considered a DLT following review by the SMC.

Patients who receive <80% of the assigned dose (less than 22 days of the 28-day cycle) of Compound A and do not experience a DLT was not considered in the assessment of the overall DLT rate for the particular dose level. Such patients may be replaced.

### Study Population

The specific eligibility criteria for selection of patients were provided. The sponsor did not grant any eligibility waivers.

### Inclusion Criteria

Each patient eligible to participate in this study must meet all of the following criteria:

Patients have voluntarily agreed to participate by giving written informed consent.

Patients with histologically or cytologically confirmed advanced or solid metastatic tumor who have experienced disease progression during or after at least 1 prior line of systemic anticancer therapy, or for which treatment was not available or not tolerated by the patient. In addition, patients must meet the following eligibility criteria for the corresponding phase of the study:
Phase 1a: Patients with a known mutation status and tumor harboring an oncogenic B-RAF or K-RAS/N-RAS mutation, or any other MAPK pathway aberrations. In Phase 1a, the study recruitment was limited to approximately one third (⅓) patients with K-RAS/N-RAS mutation, and among these patients the study recruitment were limited to approximately one third (⅓) CRC or pancreatic patients.
Phase 1b: Patients must have a known mutation status and meet one of the following criteria according to the group they were enrolled into:
Group 1: Patients with solid tumors with non-V600 B-RAF mutations including RAF fusions; or
Group 2: Patients with B-RAF V600 mutated melanoma or NSCLC, and have progressed on a B-RAF-inhibitor and/or MEK-inhibitor resistant tumors.

Patients must provide archival tumor tissue or agree to a fresh tumor biopsy for mutation and biomarkers analysis (fresh tumor biopsies were strongly recommended at Screening in patients with readily accessible tumor lesions).

Patients must have measurable disease as defined per RECIST v1.1.

Patients must be ≥18 years of age on the day of signing the ICF.

Eastern Cooperative Oncology Group (ECOG) performance status of ≤1 at Screening.

Life expectancy ≥12 weeks at Screening.

Adequate hematologic and organ function as indicated by the following laboratory values independent of transfusion within 14 days of Cycle 1 Day 1:
ANC ≥1500 cells/μL.
Platelets ≥100,000/μL.
Hemoglobin ≥8 g/dL or ≥4.96 mmol/L.
Serum CRE ≤1.5× upper limit of normal (ULN) or estimated glomerular filtration rate (GFR)>60 mL/min/1.73 m2 by Chronic Kidney Disease Epidemiology Collaboration (CKD-EPI) equation.
Serum total bilirubin ≤1.5×ULN (total bilirubin must be <3×ULN for patients with Gilbert's syndrome).

AST and ALT ≤3×ULN or ≤5×ULN for patients with liver metastases.

Female patients were eligible to enter and participate in the study if they were of:

Nonchildbearing potential (i.e., physiologically incapable of becoming pregnant), including any female who:
has had a hysterectomy.
has had a bilateral oophorectomy (ovariectomy).
has had a bilateral tubal ligation; or
is postmenopausal (total cessation of menses for ≥1 year).

Childbearing potential, has a negative serum pregnancy test at Screening (within 7 days of the first dosing of IMP), was not breastfeeding, and uses contraception before study entry and throughout the study until 180 days after the last dose of the IMP.

Male patients were eligible to enter and participate in the study if they were vasectomized or agree to use of contraception during the study treatment period and for at least 180 days after the last dose of the IMP.

### Exclusion Criteria

Patients who meet any of the following criteria must be excluded from this study:

Female patients who were pregnant or lactating.

Patients receiving cancer therapy (chemotherapy or other systemic anticancer therapies, immunotherapy, radiation therapy, or surgery) at the time of Cycle 1 Day 1.

All patients who have received prior systemic anticancer treatment within the following time frames were excluded:
Cyclical chemotherapy within a period of time that was shorter than the cycle length used for that treatment (i.e., 6 weeks for nitrosourea, mitomycin C) prior to Cycle 1 Day 1; and
Biologic therapy (i.e., antibodies), continuous or intermittent small molecule therapies, or any other investigational agents within a period of 5 times the half-life of the agent or <4 weeks (whichever was shorter) prior to Cycle 1 Day 1.

Active infection requiring systemic treatment.

Any of the following cardiovascular criteria:

Current evidence of cardiac waschemia.

Current symptomatic pulmonary embolism.

Acute myocardial infarction ≤6 months prior to Cycle 1 Day 1.

Heart failure of New York Heart Association Classification III or IV≤6 months prior to Cycle 1 Day 1.

Grade ≥2 ventricular arrhythmia ≤6 months prior to Cycle 1 Day 1.

Cerebral vascular accident (CVA) or transient waschemic attack (TIA)≤6 months prior to Cycle 1 Day 1.

Uncontrolled hypertension: systolic pressure 150 mmHg or diastolic pressure ≥100 mmHg despite anti-hypertension medications ≤28 days before Cycle 1 Day 1.

Syncope or seizure ≤28 days before Cycle 1 Day 1.

Any major surgery within 28 days prior to Cycle 1 Day 1.

Patients with toxicities (as a result of prior anticancer therapy) which have not recovered to baseline or stabilized, except for AEs not considered a probable safety risk (e.g., alopecia, neuropathy, and specific laboratory abnormalities).

History or presence of gastrointestinal disease or other condition known to interfere with the absorption, distribution, metabolism, or excretion of drugs.

Known immediate or delayed hypersensitivity reaction or idiosyncrasy to drugs chemically related to Compound A (to date, there were no known US FDA-approved drugs chemically related to Compound A).

Leptomeningeal or brain metastasis except patients with previously treated brain metastasis who were radiologically stable (imaging evidence required), asymptomatic and have been off steroids and antiseizure medications for longer than 28 days prior to Cycle 1 Day 1 were permitted.

Any active malignancy ≤2 years before Cycle 1 Day 1 except for the specific cancer under investigation in this study and any locally recurring cancer that has been treated curatively (e.g., resected basal or squamous cell skin cancer, superficial bladder cancer, or carcinoma in situ of the cervix or breast).

Any unstable, preexisting major medical condition that in the opinion of the investigator contraindicates the use of an IMP, including known human immunodeficiency virus (HIV) or active hepatitis B virus (HBV) or hepatitis C virus (HCV) infection. Patients who were hepatitis B surface antigen (HBsAg) positive or HCV antibody positive at Screening may be enrolled only if HBV DNA titers <500 IU/mL or negative HCV RNA polymerase chain reaction test, respectively.

Psychological, familial, sociological, or geographical conditions that do not permit compliance with the protocol.

Unfit for the study, in the opinion of the investigator, after medical interview, physical examination, or screening investigations.

Receiving any of the prohibited medications or required any of these medications.

Concurrent participation in another therapeutic clinical trial.

### Tumor and Response Evaluations

Tumor imaging were performed within 21 days prior to the first study treatment. Results of standard of care tests or examinations performed prior to obtaining informed consent and ≤21 days prior to the first administration of IMP may be used for the purposes of screening rather than repeating the standard of care tests. During the study, tumor imaging was performed approximately every 8 (±1) weeks in the first year and approximately every 12 (±1) weeks thereafter. Tumor assessments were required to be performed on schedule regardless of whether study treatment has been administered or held.

Tumor assessments must include computed tomography (CT) scans (with oral/IV contrast, unless contraindicated) or MRI, with preference for CT, of the chest, abdomen, and pelvis. All measurable and evaluable lesions should be assessed and documented at the Screening visit and reassessed at each subsequent tumor evaluation. The same radiographic procedure used to assess disease sites at Screening was required to be used throughout the study (i.e., the same contrast protocol for CT scans).

Magnetic resonance image of the head at baseline (≤21 days of informed consent) was required for all screened patients unless contraindicated, then a CT of head may suffice.

If a patient was known to have a contraindication to CT contrast media or develops a contraindication during the study, a non-contrast CT of the chest plus a contrast-enhanced MRI (if possible) of abdomen and pelvis should be performed.

If a CT scan for tumor assessment was performed in a positron emission tomography (PET)/CT scanner, the CT acquisition must be consistent with the standards for a full-contrast diagnostic CT scan.

Bone scans (Technetium-99m [TC-99m]) or sodium fluoride PET [NaF-PET]) should be performed at Screening if clinically indicated. If bone metastases were present at Screening and cannot be seen on CT or MRI scans afterwards, or if clinically indicated, TC-99m or NaF-PET bone scans should be repeated when a CR was suspected in the target lesion or when progression in bone was suspected.

Computer tomography scans of the neck or extremities should also be performed if clinically indicated and followed throughout the study, if there was evidence of metastatic disease in these regions at Screening. At the investigator's discretion, other methods of assessment of target lesion and nontarget lesions per RECIST v1.1 may be used.

Tumor response was assessed by the investigators using RECIST v1.1. The same evaluator should perform assessments, if possible, to ensure internal consistency across visits.

After the first documentation of response (CR or PR), confirmation of tumor response should occur at 4 weeks or later (≥4 weeks) after the first response or at the next scheduled assessment time point.

Patients who discontinue study treatment early for reasons other than disease progression (e.g., toxicity) continued to undergo tumor assessments following the original plan until the patient begins a subsequent anticancer treatment, experiences disease progression, withdraws consent, dies, or until the study terminates, whichever occurs first.

All patients' files and radiologic images must be available for source verification and for potential peer review.

### Pharmacokinetic Assessments

The following assessments were performed at a central laboratory:

Pharmacokinetic assay: plasma samples were assayed for Compound A (or possible major metabolites) concentration with use of a validated chromatography method.

Shipping, storage, and handling of samples for the PK assays were managed through a central laboratory. Instruction manuals and supply kits were provided for all central laboratory assessments. PK sampling time points were documented.

### Biomarker Assessments

Patients must have archival tumor tissue or agree to a tumor biopsy for mutation and biomarker analyses (fresh tumor biopsies were strongly recommended at screening in patients with readily accessible tumor lesions and who consent to the biopsies). For patients with readily accessible tumor lesions who consent to the biopsies, a follow-up biopsy may be taken, ideally from the same tumor lesion, for the analysis of pharmacodynamic biomarkers. For Group 2 in Phase 1b, paired fresh tumor biopsies within 8 weeks prior to first dose and at a time post-dose to assess PD biomarkers were mandatory. In addition, blood samples were collected from all patients for mutation and biomarker analysis.

Shipping, storage, and handling of tumor tissues and blood samples for the assessment of biomarkers were managed through a central laboratory. Refer to the laboratory manual for details of sample handling.

Archival tumor tissues (formalin-fixed paraffin-embedded block with tumor tissue or approximately 15 unstained slides) must be sent to the central laboratory for immunohistochemistry analysis. In addition to assessing MAPK signaling by phospho-ERK levels, mutations in B-RAF, K-RAS, N-RAS and other aberrations in the MAPK pathway as well as other markers that were related to response or clinical benefit of Compound A may also be evaluated.

For fresh biopsy specimens, acceptable samples include core needle biopsies for deep tumor tissue or excisional, incisional, punch, or forceps biopsies for cutaneous, subcutaneous, or mucosal lesions.

Tumor tissue should be of good quality based on total and viable tumor content. Fine-needle aspiration, brushing, cell pellets from pleural effusion, and lavage samples were not acceptable.

Peripheral blood samples were collected at specified timepoints as described in the schedule of assessments to be used for the longitudinal evaluation ctDNA by molecular (NGS) methodologies as pharmacodynamic biomarkers associated with response or resistance.

### Visit Windows

If the timing of a protocol-mandated study visit coincides with a holiday, weekend, or other events, the visit should be scheduled on the nearest feasible date with subsequent visits conducted according to the planned schedule.

### Statistical Methods and Sample Size Determination

The statistical analyses were performed by the sponsor or designee after the study was completed and the database was locked and released. Data were listed and summarized using SAS® Version 9.4 or higher (SAS Institute, Inc., Cary, North Carolina) per sponsor agreed reporting standards, where applicable. No missing data were imputed generally, and statistical methods were primarily descriptive in nature. Details of the statistical analyses were included in a separate statistical analysis plan (SAP).

### Statistical Analysis

### Randomization Methods

Not applicable.

### Analysis Populations

The analysis populations included:

The Safety Population included all patients who received at least 1 dose of IMP.

The Evaluable Population included all dosed patients who have evaluable disease at baseline, and at least 1 evaluable postbaseline, confirmed tumor-response assessment.

The DLT Evaluable Population included patients who received at least 80% of the assigned dose of Compound A during the dose-escalation phase (Phase 1a) or who experienced a DLT during the dose-escalation phase (Phase 1a).

The PK Population included all dosed patients for whom valid Compound A PK parameters can be estimated.

The Pharmacodynamic Population included all patients with valid Compound A PD sampling after treatment with Compound A.

### Patient Disposition

The number of patients treated, discontinued from IMP and/or the study and those with major protocol deviations were counted. The primary reason for IMP and/or study discontinuation were summarized according to the categories in the eCRF. The end of study status (alive, dead, withdrawn consent, or lost to follow-up) at the data cutoff date were summarized using the data from the eCRF.

Major protocol deviations were summarized and listed by category of deviation.

### Demographic and Other Baseline Characteristics

Demographic and other baseline characteristics were summarized in the Safety Population using descriptive statistics. Continuous variables include age, weight, vital signs, time since initial cancer diagnosis, and time since advanced/metastatic disease diagnosis; categorical variables include prior number of systemic treatments, tumor node metastasis classification of malignant tumors staging, sex, ECOG performance status, country, race, and metastatic site.

### Prior and Concomitant Medications

Concomitant medications were assigned an 11-digit code using the World Health Organization (WHO) Drug Dictionary drug codes. Concomitant medications were further coded to the appropriate Anatomical Therapeutic Chemical (ATC) code indicating therapeutic classification using the MedDRA Version 21 or higher. Prior and concomitant medications were summarized and listed by drug and drug class. Prior medications were defined as medications that stopped before the first dose of IMP. Concomitant medications were defined as medications that (1) started before the first dose of IMP and were continuing at the time of the first dose of IMP, or (2) started on or after the date of the first dose of IMP up to 30 days after the patient's last dose.

### Efficacy Analysis

The efficacy endpoints were analyzed using the Evaluable Population except for PFS which were analyzed using the Safety population.

Summaries were provided for each dose level in Phase 1a and for each patient group in Phase 1b.

For the analysis of ORR, DCR, and CBR, summary tables presenting the number and proportion of responders with the 2-sided exact (Clopper-Pearson) 95% CI for response rates were presented. Summary of each response category of the BOR (confirmed CR, confirmed PR, unconfirmed CR, unconfirmed PR, SD, or PD) were presented as well. Waterfall plots of maximum tumor shrinkage per patient were presented.

The time to event endpoints, including PFS, DOR, DSD, and overall survival were analyzed by Kaplan-Meier methods. The survival functions of the time to event endpoints were summarized for 25th percentile, median, and 75th percentile and their 95% CI. The rates of PFS, DOR, DSD and overall survival at Month 3 and every subsequent 3 months as appropriate and their 95% CI were derived based on Kaplan-Meier estimates. In addition, the graphs of Kaplan-Meier estimates of survival functions were presented.

### Safety Analyses

Safety endpoints (other than DLTs) were summarized using the Safety Population. All summaries of safety were by dose level for Phase 1a and by patient group (tumor type) for Phase 1b.

### Extent of Exposure

Treatment exposure was summarized descriptively. Measures of extent of exposure include the number of treatment cycles received, duration of exposure, and cumulative dose for the study period. The number of patients with dose delay and reason for dose discontinuation was also summarized, as appropriate.

### Adverse Events

Adverse events were coded using MedDRA, and AE data were summarized by SOC and PT. The frequency and percentage of patients by reported SOC/PT were summarized, with the number of AEs counted as well. Adverse events were also summarized by worst AE severity/grade and AE relationship to study treatment. The number of SAEs and treatment-emergent AEs (TEAEs) which led to discontinuation of study treatment was also summarized.

For Phase 1a, DLTs were summarized for the DLT Evaluable Population.

All AE summaries were restricted to TEAEs only. Treatment-emergent AEs were defined as AEs which commence, or worsen in severity, on or after the time of start of IMP administration.

A by-patient AE data listing, including verbatim term, MedDRA SOC and PT, severity, outcome and relationship to study treatment, were provided. Separate listings were generated for serious AEs and AEs leading to discontinuation of study treatment.

### Laboratory Analyses

All hematology, chemistry, and coagulation (continuous variables) parameters were summarized using descriptive statistics for all protocol scheduled time points, including change from baseline for all post-dose assessments. Number of patients with abnormal laboratory test values including their clinical significance (if available) were summarized as well. Shift tables from baseline to post-dose assessments were also generated for each safety laboratory parameter with values of “within normal limits,” “high,” and “low” used for the shift categories. Laboratory values were compared with the normal range of the laboratory, and values that fall outside of the normal ranges were flagged as: H (High) and L (Low) in the data listings.

Dipstick urinalysis results evaluation were summarized at each protocol scheduled time point. Microscopy data, if available, were listed.

### Vital Signs

Vital sign measurements and changes from baseline were summarized at each protocol scheduled time point.

### Physical Examination

Newly occurring or worsening clinically significant abnormalities identified on physical examination were captured as AEs and were not summarized or listed separately.

### 12-Lead Electrocardiogram

Descriptive statistics were calculated for 12-lead ECG parameters, including changes from baseline for all time points assessed. In addition, the overall interpretation of 12-lead ECG results was classified using frequency counts and percentages for the categories of normal, abnormal not clinically significant and abnormal clinically significant for all time points assessed, as applicable. Markedly abnormal QT interval corrected for heart rate (QTc) were also summarized as per the FDA guidance for Industry E14. Further details were provided in the SAP.

### Eastern Cooperative Oncology Group Performance Status

Shifts from baseline in ECOG performance status were summarized descriptively using frequency counts and percentages for each dose level/treatment group at all the protocol scheduled time points.

### Pharmacokinetic Analysis

Pharmacokinetic data were analyzed using the PK Population. Compound A PK variables (i.e., AUClast, AUC0-∞, Cmax, Tmax, t1/2, apparent clearance and Vz/F) were calculated after single dosing in Phase 1a. In addition, AUClast, ss, AUCtau, Cmax,ss, and tmax,ss were calculated in Phase 1b. The PK parameters were calculated as appropriate using noncompartmental methods and summary statistics were provided. Compound A (or possible major metabolites) plasma concentration data and PK parameters were tabulated and summarized for Day 1 of Cycle 1, Day 1 of Cycle 2 and at steady state (Phase 1b). Descriptive statistics included means, medians, minimums, maximums, standard deviation, coefficient of variation (CV), geometric mean, and geometric CV as appropriate. Mean plasma concentrations were also plotted against time for each dose level. Additional PK analyses may be conducted as appropriate.

Exposure-response (efficacy or safety endpoints) analysis may be carried out if supported by data. Results of such analyses may be reported separately from the clinical study report (CSR).

### Other Exploratory Analyses

Biomarker data were analyzed using the biomarker evaluable and pharmacodynamic population. Summary statistics were provided for pharmacodynamic biomarkers, including but not limited to mutation, transcription and phosphorylation profiles of MAPK pathway signaling in both paired biopsies and longitudinal blood samples. An exploratory analysis on a potential correlation of these PD markers with the dose, pharmacokinetics, safety, and antitumor activity were performed as appropriate. The primary predictive biomarker analysis was based on a subset of the patients with aberrations in the MAPK pathway such as oncogenic B-RAF, K-RAS, N-RAS, and NF-1 mutations. Exploratory analyses of other candidate predictive biomarkers were conducted similarly. Depending on the data available, the analysis for biomarkers were descriptive in nature. Details were provided in the SAP, as applicable.

### Sample Size Consideration

No formal statistical calculation of sample size was used, and the sample size was empirical. The study plans to enroll approximately 30 to 60 patients.

Phase 1a: Approximately 18 to 30 patients for the dose escalation until MTD and/or RP2D determination

Phase 1b: Approximately 30 patients for expansion in 2 selected groups. Each group were evaluated separately and can be closed due to futility or clinical efficacy based on statistical evaluation or insufficient patient recruitment. If promising preliminary efficacy results have been observed in 1 of the arms after treating all planned patients (i.e., higher ORR or longer PFS), more patients could be added to the arm to further assess the efficacy before moving into Phase 2/3 clinical development.

### Interim Analyses

No formal inference related interim analysis were conducted. However, the sponsor may request interim descriptive analyses of safety and efficacy data. The interim analyses were for the purpose of safety monitoring and planning of future studies and not affected the conduct of this study. Efforts were made to keep the bias possibly associated with interim analyses to a minimal level.

Summary of all cohorts is provided in Table 3. 1 patient in cohort 1 was not evaluable for DLT due to rapid disease progression.

Summary of cohort 5 is provided in Table 4 with the following results:
4 Patients enrolled in the Cohort
4 patients have completed the DLT period with no DLTs

Dose Discontinuation or Delay:
102-012 discontinued disease progression.
201-010 discontinued disease progression.
202-012 dose delay, decreased ejection fraction; timing TBC.
102-012 had CR target lesion week 16.
2 SAEs (nil additional from previous meeting).
Fever, Grade 2, related.
Bowel Obstruction, Grade 3, not related.
2 patients dose delayed (nil additional from previous).

Tumor response:
101-010 PR week 8, SD week 16; 102-012 PR week 8, CR week 16; 202-012 SD week 8.

Summary of cohort 6 is provided in Table 5 with the following results:
4 Patients enrolled in the Cohort.
2 patients completed the DLT period with 1 DLT.
1 patient discontinued in Cycle 1, SAE (fever & angioedema).

8 SAEs:
201-011: AF, Grade 3, related.
202-014: Rhabdomyolysis ×2, Grade 2 and Grade 3 (few days later), related; pain in lower extremities, Grade 3, related; acneiform rash, Grade 2, related.
203-007: Febrile syndrome ×2, Grade 2 and Grade 3, related; Angioedema, Grade 2, related.
201-011 dose delayed as a result of AF Grade 3, related.

Tumor response:
1 patient evaluable, 201-011: SD week 8.
Preliminary PK parameters and data are listed in Table 6.
Parameters listed as Mean (% CV)
Tₘₐₓ reported as median (min, max)
Accumulation ratio R_{acc} based on AUC8h
Patient 101-007 was excluded from C2D1 of cohort 2 (10 mg) due to dose reduction.
Patient 101-011 was excluded from summary statistics of CID1 of cohort 6 (60 mg) due to AQL samples.
Patient 201-011 C2D1 parameters excluded from summary statistics of C2D1 of cohort 6 due to extended dose interruption.

Overall Summary of Adverse Events are provided in Table 7.
201-003 Respiratory failure—not related to IP
FEVER, Gr 1, related, recovered.
a: figures updated from last safety meeting.

### Adverse Event Summary All Cohorts

Grade 5:
1 event for patient 201-003 in cohort 2, Respiratory failure not related.
Grade 4:
1 event for patient 201-005 in cohort 3, Hypercalcaemia not related. Grade 3:
55 events in 22 patients, 18 related to study drug.
37 SAEs in 19 patients.
11 non-serious in 9 patients.
Grade 2:
107 events in 23 patients, 38 related to study drug.
18 SAEs in 9 patients.
86 non-serious in 22 patients.
Grade 1:
241 events in 36 patients, 84 related to study drug.
6 SAEs in 3 patients.
230 non-serious in 35 patients.

### Adverse Event Summary Cohort 6

Grade 3:
8 events in 3 patients.
4 SAEs:
201-011 AF related;
202-014 pain in lower extremities, related;
202-014 rhabdomyolysis, related;
203-007 febrile syndrome, related.
Grade 2:
18 events in 2 patients, 15 related to study drug.
4 SAEs in 2 patients.
12 non-serious in 2 patients.
Grade 1:
19 events in 3 patients.
15 non-serious events in 3 patients. patient 101-011 (×3), 202-014 (×5), 203-007 (×7).
12 related to study drug.

* Note some AEs have not been designated either serious or non-serious in data extract.

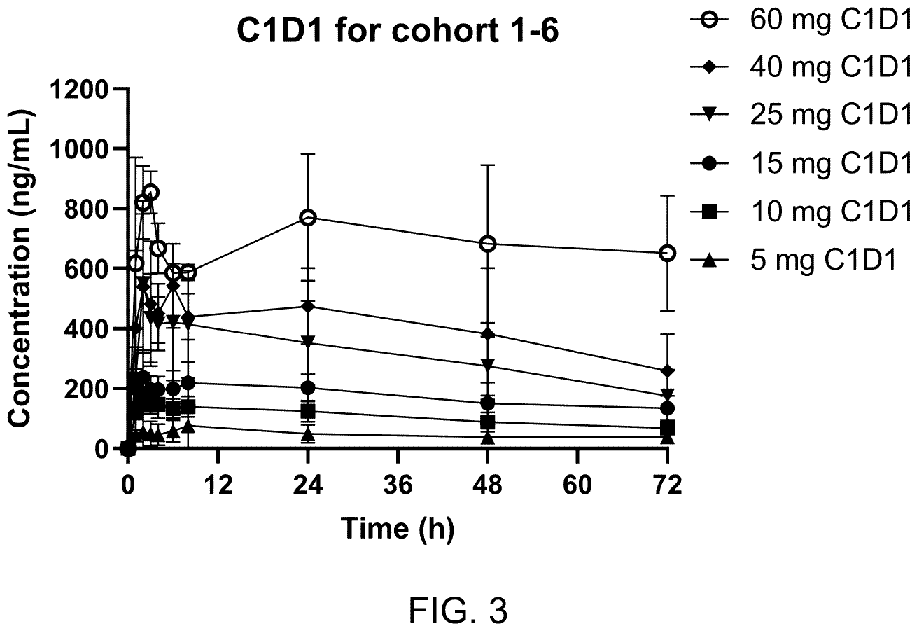

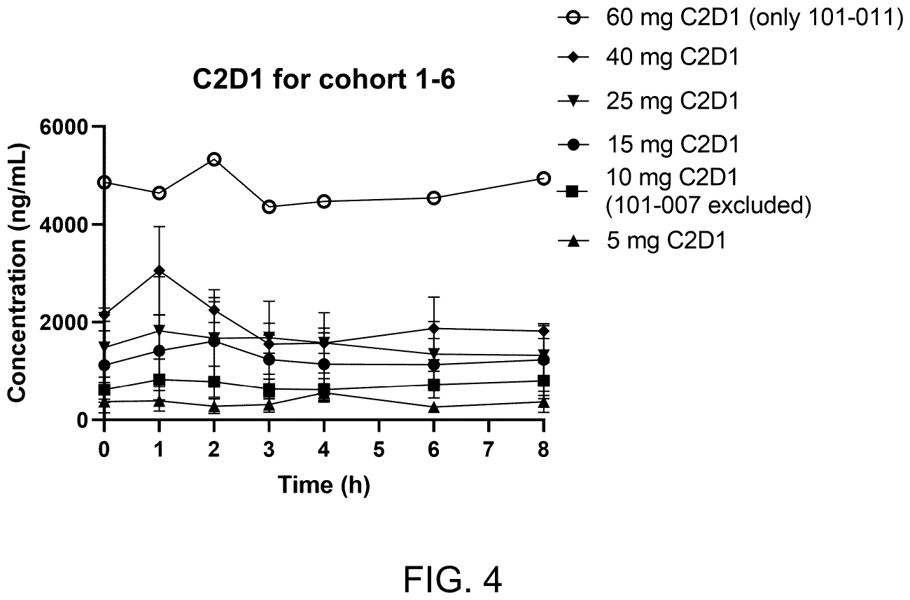

FIG. 3 and FIG. 4 provide the preliminary mean PK profiles for all cohorts for C1D1 and C2D1, respectively.

TABLE 3
Summary of All Cohorts

| | | | | DLT | | | |
|---|---|---|---|---|---|---|---|
| Cohort | Status | Study Drug | Enrolled | Evaluable | DLTs | Active | DLT Detail |
| 1 | Cleared | 5 mg PO QD | 4 | 3 | 0 | 0 | 1 patient in cohort 1 was not evaluable for |
| | | | | | | | DLT due to rapid disease progression. |
| 2 | Cleared | 10 mg PO QD | 12 | 8 | 2 Pts: | 3 | DLT: G3 maculopapular rash |
| | | | | | 202-002 | | DLT: G3 maculopapular rash |
| | | | | | 101-007 | | 3 Pts not eval for DLT due to <80% IP |
| | | | | | | | taken |
| 3 | Cleared | 15 mg PO QD | 7 | 7 | 0 | 3 | — |
| 4 | Cleared | 25 mg PO QD | 7 | 5 | 0 | 4 | 1 Pts not eval for DTL due to <80% IP |
| | | | | | | | taken |
| | | | | | | | 1 patient withdrew consent during C1D1 |
| | | | | | | | 1 patient discontinued due to PD |
| 5 | Ongoing | 40 mg PO QD | 4 | 4 | 0 | 3 | |
| 6 | Ongoing | 60 mg PO QD | 4 | 2 | 1 | 3 | 1 Pt discontinued, and not DLT evaluable |
| | | | | | | | Fever and Angioedema. 2nd patient |
| | | | | | | | insufficient IP over DLT period. |

TABLE 4
Summary of Cohort 5

| Subject | | | DLT | | | |
|---|---|---|---|---|---|---|
| Number | Cancer | Mutation | Evaluable? | DLT? | Status | Results |
| 101-010 | Melanoma | BRAF V600E | Yes | No | Completed DLT | PR week 8; |
| | | | | | period | SD week 16 |
| | | | | | successfully | |
| 201-010 | Colorectal | BRAF K499N | Yes | No | Completed DLT | |
| | Adenocarcinoma | and BRAF | | | period | |
| | | L505H | | | successfully | |
| 102-012 | Melanoma | BRAFV600E | Yes | No | Completed DLT | PR week 8; |
| | | | | | period | CR week 16 |
| | | | | | successfully | |
| 202-012 | Lung | BRAF V600E | Yes | No | Completed DLT | SD week 8 |
| | Adenocarcinoma | | | | period | |
| | | | | | successfully | |

TABLE 5
Summary of Cohort 6

| Subject | | | DLT | | | |
|---|---|---|---|---|---|---|
| Number | Cancer | Mutation | Evaluable? | DLT? | Status | Results |
| 101-011 | Endometrial cancer | BRAF Class II | Yes | No | On treatment | |
| | | (K601E) | | | | |
| 202-014 | Intrahepatic | BRAF V600E | Yes | Yes | On treatment | |
| | cholangiocarcinoma | | | | | |
| 201-011 | Melanoma | BRAF | No | No | On treatment | |
| | | pathogenic | | | | |
| | | fusion AGK- | | | | |
| | | BRAF | | | | |
| 203-007 | Melanoma | BRAF | No | No | Discontinued | SAE Fever and |
| | Adenocarcinoma | | | | | Angioedema |

TABLE 6
preliminary PK parameters and data

| | | Cycle 1 Day 1 (C1D1) | | | Cycle 2 Day 1 (C2D1) | | | |
|---|---|---|---|---|---|---|---|---|
| | | Cₘₐₓ | AUC_{8 h} | AUC_{24 h} | Cₘₐₓ | AUC_{8 h} | Tₘₐₓ | |
| Dose | PID | ng/mL | ng · h/mL | ng · h/mL | ng/ml | ng · h/mL | h | R_{acc} |
| 5 mg | Mean | 105 (64%) | 414 (68%) | 1380 (60%) | 451 (61%) | 2660 (51%) | 1 (1, 4) | 17 (117%) |
| 10 mg | Mean | 197 (19%) | 1060 (22%) | 3140 (21%) | 973 (41%) | 5720 (37%) | 1 (0, 8) | 6 (49%) |
| 15 mg | Mean | 274 (35%) | 1530 (30%) | 4780 (26%) | 1660 (46%) | 9930 (53%) | 2 (1, 3) | 7 (60%) |
| 25 mg | Mean | 593 (36.5%) | 3090 (31.7%) | 9170 (36.5%) | 2110 (40.0%) | 12300 (46.5%) | 1 (0, 4) | 4 (23%) |
| 40 mg | 101-010 | 692 | 3850 | 12000 | 3600 | 15600 | 1 | 4.05 |
| | 102-012 | 789 | 4510 | 12300 | 3550 | 18300 | 1 | 4.06 |
| | 201-010 | 415 | 2470 | 8460 | 2410 | 13400 | 2 | 5.43 |
| | 202-012 | 896 | 5040 | 15700 | 2300 | 15400 | 3 | 3.06 |
| | Mean | 698 (29.5%) | 3960 (28.0%) | 12100 (24.3%) | 2970 (23.8%) | 15700 (12.8%) | 1.5 (1, 3) | 4 (23.5%) |
| 60 mg | 101-011** | 878 | 5630 | 18900 | 5330 | 37500 | 2 | 6.7 |
| | 201-011^{##} | 957 | 5510 | 17600 | 5230 | 31300 | 2 | 5.7 |
| | 202-014 | 804 | 4340 | 13100 | | | | |
| | Mean | 881 (12.3%) | 4929 (16.8%) | 15363 (20.8%) | 5330 | 37500 | 2 | TBD |

TABLE 7
Overall Summary of Adverse Events

| | 1 | 2 | 3 | 4 | 5^{a} | 6 | |
|---|---|---|---|---|---|---|---|
| Cohort number | (5 mg | (10 mg | (15 mg | (25 mg | (40 mg | (60 mg | |
| (Compound A dose) | PO QD) | PO QD) | PO QD) | PO QD) | PO QD) | PO QD) | Total |
| Treated Subjects | 4 | 12 | 7 | 7 | 4 | 4 | 34 |
| Pts with ≥1 DLT | 0 | 2 | 0 | 0 | 0 | 1 | 3 |
| AE, any cause | 8 | 101 | 13 | 68 | 39 | 45 | 274 |
| AE, related | 1 | 44 | 4 | 11 | 24 | 35 | 119 |
| AE any cause, Grade 3 | 1 | 15 | 0 | 11 | 0 | 8 | 35 |
| AE, related, Grade 3 | 0 | 7 | 0 | | 0 | 8 | 15 |
| SAE, any cause | 1 | 18 | 0 | 10 | 2 | 8 | 39 |
| SAE, related | 0 | 7 | 0 | 0 | 1** | 8 | 16 |
| Death | 0 | 1* | 0 | 0 | 0 | 0 | 1 |
| Count Of Subjects | 4 | 12 | 7 | 7 | 4 | 4 | 38 |
| Patients with ≥1 AE that | 0 | 1 | 0 | 0 | 0 | 0 | 1 |
| required treatment | | | | | | | |
| modification | | | | | | | |
| Patients with Dose delay | 0 | 9 | 2 | 2 | 2* | 0 | 15 |
| Dose reduction | 0 | 1 | 0 | 0 | 1 | 1 | 3 |
| Permanent | 2 | 5 | 0 | 0 | 0 | 1 | 8 |
| discontinuation | | | | | | | |

Compound A is a highly potent RAF kinase inhibitor with equal potency against RAF monomer & dimer.

Compound A has potentials in targeting non-V600E BRAF mutations; and addressing resistance to 1st generation RAF/MEKi.

Compound A was well tolerated at 5 mg, 10 mg, 15 mg, 25 mg, and 40 mg QD in Cohort 5.

Results observed in Cohort 5 are following:

Exposure of Compound A across cohorts was generally proportional to the increase in the dose.

2 patients evaluable for tumor response had a PR at Week 8.

Cohort decision and the next steps:

Compound A dose is escalated to 60 mg QD in Cohort 6 (50% increase from Cohort 5) to be administered as 6 (six) Compound A capsules of 10 mg each.

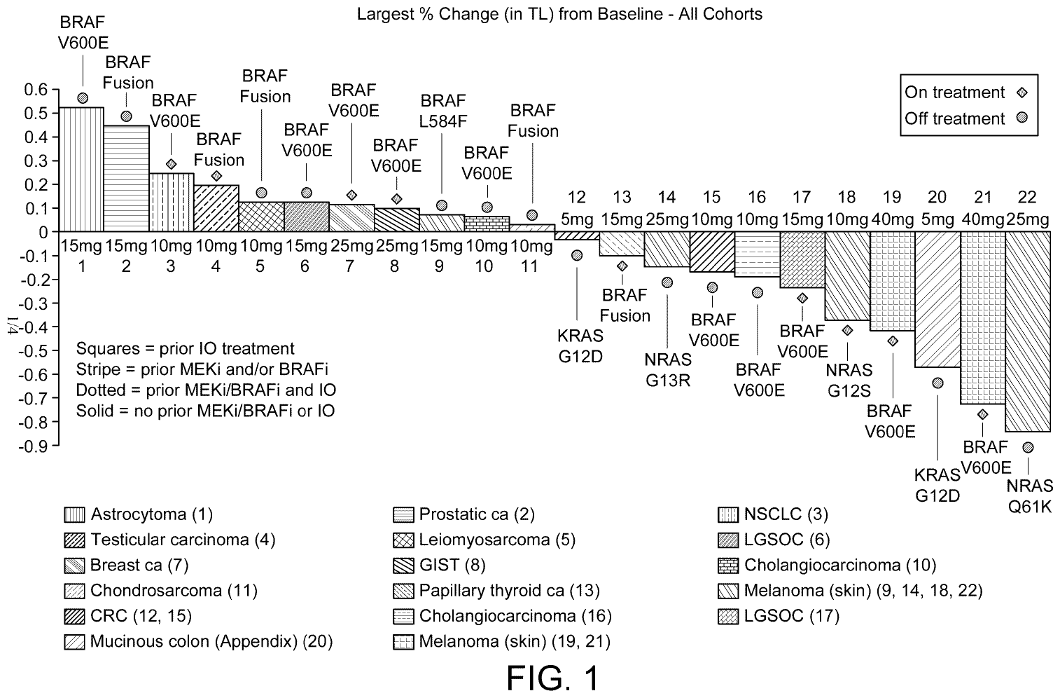

FIG. 1 describes the best change (%) from baseline in sum of diameters per RECIST 1.1 (efficacy in total 22 evaluable patients). FIG. 1 showed the following:
5/22 (22.7%) overall response rate.
4/5 (80%) responders have melanoma.
7/22 (31.8%) efficacy evaluable patients have melanoma.
7/7 (100%) melanoma patients showed stable disease, partial responses, or complete responses.
1 melanoma patients showed complete responses (1 CT readout).
3 melanoma patients showed partial responses.
3 melanoma patients showed stable disease.
2/2 (100%) CRC patients showed stable disease.

### Materials and Methods

Eligible pts were ≥18 yrs old with ECOG 0-1 and had solid tumors harboring

MAPK pathway alterations. Dose-escalation and cohort-size decisions were made using the mTPI-2 design. The starting dose was 5 mg QD.

Treatment emergent adverse events (TEAEs) were graded per NCI CTCAE v5.0. Tumor responses were assessed by investigators using RECIST v1.1.

### Results

42 pts were treated across 6 cohorts (5-60 mg QD). The median age was 60 yrs; pts had received a median of 3 prior lines of treatment.

98% pts had TEAEs; 76% had treatment-related (TR) AEs. The most commonly reported TRAEs (≥10%) were skin-related (69%), and fever and diarrhea (both 12%). 13 pts had Gr ≥3 TRAEs (≥2 pts): platelet count decreased (3), rash acneiform (2), and ALT and AST elevations (2 each). Dose reductions occurred in 4 pts due to rhabdomyolysis (2), angioedema (1), and AST elevation (1). Dose interruptions due to AEs occurred in 36% of pts. 40 mg QD was determined as the MTD.

Serious TEAEs occurred in 71% of pts; 29% were drug-related. DLTs occurred in 6/42 pts (2 at 10 mg, 1 at 40 mg, and 3 at 60 mg).

Preliminary PK results showed exposure generally increased dose-proportionally. The Tmax occurred at ˜2 h; Compound A had a long terminal half-life and 7.4-fold average accumulation in exposure at steady-state.

100% pts were efficacy evaluable. The disease control rate (DCR) was 33% with 1 CR, 5 cPR, 2 uPR and 8 SD≥24 weeks. Objective responders included BRAF V600E melanoma pts who progressed on prior BRAF/MEK and checkpoint inhibitors (2; 1 CR, 1 PR), 1 NRAS G12S melanoma, 1 NRAS Q61K melanoma, 1 BRAF V600E LGSOC, 1 BRAF K601E/PIK3CA endometrial cancer, 1 BRAF V600E cholangiocarcinoma, and 1 KRAS G12D appendiceal cancer.

Analysis of circulating tumor DNA samples from a subset of melanoma pts showed that maximum reduction of variant allele frequency (VAF) of detected mutations was 99.3% (range: 98.7-99.9%) across evaluated subjects following Compound A treatment, corresponding to clinical response.

### Conclusions

Compound A has a manageable safety profile and a generally dose-proportional PK. Antitumor activity was observed in pts with no approved targeted therapy options. The safety and early efficacy profile of Compound A supported further investigation in selected MAPK-altered tumors.

42 patients were treated across six dose levels (5-60 mg daily). Patients were heavily pre-treated, having received a median three prior lines of therapy (range 1-9), including standard of care immunotherapy and targeted therapy regimens. Results demonstrated that Compound A had a manageable safety profile, with adverse event findings consistent with other MAPK pathway inhibitors. The most common treatment-related adverse events (>15%) were rash acneiform (33%), rash maculopapular (24%), and fever (17%). The 40 mg once daily dose was determined to be the maximum tolerated dose of Compound A. In addition, encouraging anti-tumor activity was observed in heavily pretreated patients, with an objective response rate of 18% (6 confirmed responses including one complete response in 33 efficacy evaluable patients). The disease control rate was 79% and clinical benefit rate was 42%. Objective responders include patients with tumors harboring BRAF V600E that had progressed on prior BRAF/MEK inhibitors with or without checkpoint inhibitor treatment, BRAF Class II mutation, BRAF fusion, NRAS and KRAS mutations. Median time on treatment was approximately 5 months (range: 1.9-23.6 months) and 9 patients remain on treatment. These data supported the advancement of Compound A into the Phase 1b dose expansion portion of the study.

These data supported the ongoing investigation of Compound A in defined cohorts, including BRAF V600 tumors that have progressed after prior BRAF and/or MEK inhibitor treatment, solid tumors with BRAF class II mutations and BRAF fusions, and NRAS mutant melanoma. These patients had very limited treatment options.

### Example 2

General properties of the solid form of Compound A used are provided in Table 8.

TABLE 8
General Properties of the solid form of Compound A used

| Property | Description |
|---|---|
| Appearance | White to off-white powder |
| Solubility | Low solubility in water; high solubility |
| | in dimethyl sulfoxide (DMSO) and |
| | dimethylacetamide (DMA) |
| Permeability | High (Papp > 10 × 10⁻⁶ cm/s) |
| pH value | 5.76 |
| Hygroscopicity | Hygroscopic |
| Thermogravimetric | A total of 2.23% weight loss |
| Analysis (TGA) | (2 weight losses, 1.482% and 0.7509%) |
| | before sample decomposition |
| Differential scanning | Multiple thermal events |
| calorimetry (DSC) | |
| pKa | 5.28 |
| Log P | >4.457 |
| Specific rotation | [α]²⁰D = −11.63° (c 0.3018 g/100 mL, |
| | acetonitrile, wavelength: 589 nm) |
| Absolute configuration | There are 3 chiral centers (C1S, C1aS, C6bS) |
| Solid form | Form I of Compound A in Example 10 of |
| | WO2020151756 |

A number of references have been cited, the disclosures of which were incorporated herein by reference in their entirety.

What was claimed was:

1. A method of treating a cancer in a subject in need thereof, comprising administering to the subject Compound A (1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl)urea) having the following structure: 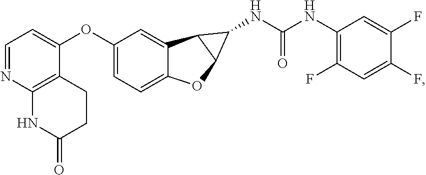 [SMILES: O=C1CCc2c(Oc3ccc4c(c3)[C@H]3[C@H](NC(=O)Nc5cc(F)c(F)cc5F)[C@H]3O4)ccnc2N1]
or a pharmaceutically acceptable salt, tautomer, stereoisomer, enantiomer, isotopologue, solvate, or prodrug thereof.

2. The method of claim 1, wherein the cancer was selected from the group consisting of colorectal cancer, pancreatic cancer, melanoma, non-small cell lung cancer, brain cancer, lung cancer, kidney cancer, bone cancer, liver cancer, bladder cancer, breast, head and neck cancer, ovarian cancer, skin cancer, adrenal cancer, cervical cancer, lymphoma, thyroid tumor, and their complications; preferably melanoma, and non-small cell lung cancer.

3. The method of claim 2, wherein the cancer was characterized by a mutation in a gene selected from the group consisting of RAS, NRAS, KRAS, RAF, BRAF, CRAF, ARAF, and their combination thereof.

4. The method of claim 3, wherein the cancer was characterized by a mutation in a gene selected from the group consisting of RAS, NRAS, KRAS, RAF, BRAF, and their combination thereof.

5. The method of claim 4, wherein the cancer was characterized by a mutation in a gene selected from the group consisting of NRAS, KRAS, BRAF, and their combination thereof.

6. The method of claim 2, wherein the cancer was characterized by:
(i) a mutation in a gene selected from the group consisting of ARAF, BRAF, RAF1, KRAS, HRAS, NF1, MAP2K1, MAP2K2, MAPK1, and their combination thereof;
(ii) a mutation selected from the group consisting of BRAF N20T, BRAF A33T, BRAF S36A, BRAF V47 G393del, BRAF V47_G327del, BRAF V47_D380del, BRAF V47_M438del, BRAF N49I, BRAF M53I, BRAF L64I, BRAF G69S, BRAF A81_D380del, BRAF A81_M438del, BRAF G104E, BRAF T119S, BRAF P141L, BRAF S151A, BRAF P162S, BRAF V169_G327del, BRAF V169_D380del, BRAF R188T, BRAF Q201H, BRAF G203_G393del, BRAF K205Q, BRAF V226L, BRAF E228V, BRAF R239Q, BRAF T241P, BRAF T241M, BRAF L245F, BRAF A246P, BRAF F247L, BRAF Q257R, BRAF Q257H, BRAF G258V, BRAF F259L, BRAF Q262R, BRAF H269Y, BRAF R271H, BRAF E275K, BRAF D287H, BRAF F294L, BRAF T310I, BRAF A320T, BRAF 1326V, BRAF P341S, BRAF R347*, BRAF P348T, BRAF S363F, BRAF S364L, BRAF P367S, BRAF P367R, BRAF P367L, BRAF D380H, BRAF R389C, BRAF T401I, BRAF A404Cfs*9, BRAF P407L, BRAF S419Y, BRAF G421V, BRAF R444W, BRAF D448Y, BRAF D449Y, BRAF W450*, BRAF W450L, BRAF E451K, BRAF E451Q, BRAF P453T, BRAF V459L, BRAF R462E, BRAF R462K, BRAF R462I, BRAF 1463T, BRAF 1463S, BRAF G464I, BRAF G464R, BRAF G464E, BRAF G464A, BRAF G464V, BRAF S465D, BRAF S465E, BRAF S465A, BRAF G466R, BRAF G466E, BRAF G466A, BRAF G466V, BRAF S467A, BRAF S467L, BRAF F468C, BRAF G469L, BRAF G469del, BRAF G469S, BRAF G469R, BRAF G469E, BRAF G469A, BRAF G469V, BRAF T470K, BRAF V471I, BRAF V471F, BRAF Y472dup, BRAF Y472S, BRAF Y472C, BRAF G478C, BRAF K483E, BRAF K483M, BRAF L485_P490del, BRAF L485Y, BRAF L485_P490delinsY, BRAF L485S, BRAF L485W, BRAF L485F, BRAF L485_P490delinsF, BRAF N486_Q494del, BRAF N486del, BRAF N486_T488del, BRAF N486_T491del, BRAF N486_L495del, BRAF N486D, BRAF N486_V487del, BRAF N486_P490del, BRAF N486_A489delinsK, BRAF N486_T491delinsK, BRAF V487_P490del, BRAF V487_P492delinsA, BRAF T488_P492del, BRAF T488_Q493delinsK, BRAF A489_P490del, BRAF P490del, BRAF P490_Q494del, BRAF K499E, BRAF K499N, BRAF E501K, BRAF E501G, BRAF V504_R506dup, BRAF V504I, BRAF L505F, BRAF L505H, BRAF R509G, BRAF R509H, BRAF L514V, BRAF M517I, BRAF Q524L, BRAF L525R, BRAF T529M, BRAF T529N, BRAF T529I, BRAF W531C, BRAF G534D, BRAF Y538H, BRAF R558Q, BRAF G563D, BRAF H568D, BRAF H574N, BRAF H574Y, BRAF H574Q, BRAF N581D, BRAF N581Y, BRAF N581T, BRAF N581S, BRAF N581I, BRAF N581K, BRAF 1582M, BRAF F583C, BRAF L584F, BRAF H585Y, BRAF E586K, BRAF D587A, BRAF D587G, BRAF D587E, BRAF V590I, BRAF V590G, BRAF 1592V, BRAF 1592M, BRAF G593D, BRAF D594N, BRAF D594H, BRAF D594Y, BRAF D594_T599dup, BRAF D594A, BRAF D594G, BRAF D594V, BRAF D594E, BRAF F595L, BRAF F595S, BRAF G596S, BRAF G596R, BRAF G596C, BRAF G596D, BRAF G596V, BRAF L597S, BRAF L597V, BRAF L597Q, BRAF L597P, BRAF L597R, BRAF A598T, BRAF A598S, BRAF A598V, BRAF A598_T599insARC, BRAF A598_T599insV, BRAF T599dup, BRAF T599A, BRAF T599K, BRAF T599R, BRAF T599I, BRAF T599_V600insTT, BRAF T599_V600insS, BRAF T599_V600insETT, BRAF T599_V600insEAT, BRAF V600_K601delinsEN, BRAF V600_S605delinsEISRWR, BRAF V600K, BRAF V600R, BRAF V600Q, BRAF V600dup, BRAF V600delinsYM, BRAF V600M, BRAF V600L, BRAF V600D, BRAF V600_K601delinsE, BRAF V600E, BRAF V600A, BRAF V600G, BRAF K601del, BRAF K601Q, BRAF K601E, BRAF K601_W604del, BRAF K601T, BRAF K601I, BRAF K601_S602delinsNT, BRAF K601N, BRAF S602T, BRAF S602Y, BRAF S602F, BRAF R603*, BRAF W604del, BRAF W604R, BRAF W604G, BRAF S605A, BRAF S605F, BRAF S605E, BRAF S605G, BRAF S605N, BRAF S605I, BRAF G606W, BRAF G606E, BRAF G606A, BRAF G606V, BRAF S607P, BRAF S607F, BRAF H608R, BRAF Q609E, BRAF Q609L, BRAF Q609H, BRAF E611D, BRAF L613F, BRAF G615R, BRAF L618F, BRAF W619R, BRAF S637*, BRAF V639I, BRAF E648Q, BRAF Y656D, BRAF R671Q, BRAF P676S, BRAF L678I, BRAF V681I, BRAF E695K, BRAF K698R, BRAF L711F, BRAF A712T, BRAF R719S, BRAF H725Y, BRAF A728V, BRAF P731T, BRAF P731S, BRAF P731L, BRAF A762E, BRAF A762V, and their combination thereof;
(iii) a mutation selected from the group consisting of KIAA1549-BRAF fusion, BCASI-BRAF fusion, CCDC6-BRAF fusion, CDC42BPB-BRAF fusion, FAM131B-BRAF fusion, FXRI-BRAF fusion, GIT2-BRAF fusion, KLHL7-BRAF fusion, RNF130-BRAF fusion, TMEM106B-BRAF fusion, MKRN1-BRAF fusion, AGAP3-BRAF fusion, AGK-BRAF fusion, AKAP9-BRAF fusion, ARMC10-BRAF fusion, CULI-BRAF fusion, GTF2I-BRAF fusion, PAPSSI-BRAF fusion, PCBP2-BRAF fusion, PPFIBP2-BRAF fusion, SND1-BRAF fusion, TRIM24-BRAF fusion, ZKSCAN1-BRAF fusion, SEPT3-BRAF fusion, and their combination thereof; or
(iv) a mutation selected from the group consisting of NRAS G12A, NRAS G12C, NRAS G12D, NRAS G12N, NRAS G12P, NRAS G12R, NRAS G12S, NRAS G12V, NRAS G12Y, NRAS G13A, NRAS G13C, NRAS G13D, NRAS G13E, NRAS G13N, NRAS G13R, NRAS G13S, NRAS G13V, NRAS A18T, NRAS I24N, NRAS P34L, NRAS Y40*, NRAS Q43*, NRAS T50I, NRAS T58I, NRAS A59G, NRAS A59D, NRAS A59T, NRAS G60E, NRAS G60R, NRAS Q61E, NRAS Q61H, NRAS Q61H, NRAS Q61K, NRAS Q61L, NRAS Q61L, NRAS Q61P, NRAS Q61R, NRAS Q61R, NRAS Q61R, NRAS Q61*, NRAS E63K, NRAS Y64D, NRAS S65C, NRAS R68S, NRAS S89A, NRAS G115Efs*46, NRAS E132K, NRAS K135N, NRAS A146P, NRAS A146T, NRAS A146V, NRAS E162*, and their combination thereof.

7. The method of claim 2, wherein the cancer was characterized by a mutation selected from the group consisting of NRAS Q61R, NRAS Q61K, NRAS Q61L, NRAS G12S, NRAS G13R, KRAS G12A, KRAS G12C, KRAS G12D, KRAS G12V, BRAF V600E, BRAF fusion, and their combination thereof.

8. The method of claim 7, wherein the cancer was characterized by a mutation selected from the group consisting of NRAS Q61R, NRAS Q61K, NRAS Q61L, KRAS G12D, KRAS G12V, BRAF V600E, BRAF fusion, and their combination thereof.

9. The method of claim 8, wherein the cancer was characterized by a mutation selected from the group consisting of NRAS Q61R, NRAS Q61K, NRAS Q61L, KRAS G12D, KRAS G12V, and their combination thereof.

10. The method of claim 2, wherein the cancer was characterized by other MAPK pathway genomic aberration.

11. The method of claim 2, wherein the cancer is melanoma.

12. The method of claim 11, wherein the melanoma is cutaneous melanoma.

13. The method of claim 11, wherein the melanoma is metastatic melanoma.

14. The method of claim 2, wherein the cancer is non-small cell lung cancer.

15. The method of claim 2, wherein the cancer is colorectal cancer.

16. The method of claim 2, wherein the cancer is ovarian cancer.

17. The method of claim 2, wherein Compound A or a pharmaceutically acceptable salt, tautomer, stereoisomer, enantiomer, isotopologue, solvate, or prodrug thereof is administered one to three times a day.

18. The method of claim 2, wherein Compound A or a pharmaceutically acceptable salt, tautomer, stereoisomer, enantiomer, isotopologue, solvate, or prodrug thereof is administered once a day.

19. The method of claim 2, wherein Compound A or a pharmaceutically acceptable salt, tautomer, stereoisomer, enantiomer, isotopologue, solvate, or prodrug thereof is administered twice a day.

20. The method of claim 2, wherein Compound A or a pharmaceutically acceptable salt, tautomer, stereoisomer, enantiomer, isotopologue, solvate, or prodrug thereof is administered three times a day.

21. The method of claim 2, wherein Compound A or a pharmaceutically acceptable salt, tautomer, stereoisomer, enantiomer, isotopologue, solvate, or prodrug thereof is administered from about 5 mg to about 600 mg per day per day.

22. The method of claim 21, wherein Compound A or a pharmaceutically acceptable salt, tautomer, stereoisomer, enantiomer, isotopologue, solvate, or prodrug thereof is administered at about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 115 mg, or about 120 mg per day per day.

23. The method of claim 22, wherein Compound A or a pharmaceutically acceptable salt, tautomer, stereoisomer, enantiomer, isotopologue, solvate, or prodrug thereof is administered at about 40 mg or about 60 mg per day.

24. The method of claim 23, wherein Compound A or a pharmaceutically acceptable salt, tautomer, stereoisomer, enantiomer, isotopologue, solvate, or prodrug thereof is administered at about 40 mg per day.

25. The method of claim 23, wherein Compound A or a pharmaceutically acceptable salt, tautomer, stereoisomer, enantiomer, isotopologue, solvate, or prodrug thereof is administered at about 60 mg per day.

26. The method of claim 2, wherein the method provides a plasma Compound A AUC₈ₕ between about 2,128 ng*h/ml and about 3,192 ng*h/ml in the subject.

27. The method of claim 2, wherein the method provides a plasma Compound A AUC₈ₕ between about 4,576 ng*h/ml and about 6,864 ng*h/ml in the subject.

28. The method of claim 2, wherein the method provides a plasma Compound A AUC₈ₕ between about 7,944 ng*h/ml and about 11,916 ng*h/ml in the subject.

29. The method of claim 2, wherein the method provides a plasma Compound A AUC₈ₕ between about 9,840 ng*h/ml and about 14,760 ng*h/ml in the subject.

30. The method of claim 2, wherein the method provides a plasma Compound A AUC₈ₕ between about 12,640 ng*h/ml and about 18,960 ng*h/ml in the subject.

31. The method of claim 2, wherein the method provides a plasma Compound A AUC₈ₕ between about 30,000 ng*h/ml and about 45,000 ng*h/ml in the subject.

32. The method of claim 2, wherein the subject achieves a stable disease, a partial response, or a complete response.

33. The method of claim 2, wherein the subject achieves a partial response or a complete response.

34. The method of claim 2, wherein the subject achieves a complete response.

35. The method of claim 2, wherein the subject does not experience a progressive disease.

36. The method of claim 2, wherein the subject achieves a stable disease.

37. The method of claim 2, wherein the subject achieves a partial response.
